(19) United States
(12) Patent Application Publication
(10) Pub. No.: US 2020/0085719 A1
(43) Pub. Date: Mar. 19, 2020

(54) COSMETIC COMPOSITION COMPRISING WATER, A SILICONE RESIN AND A NON-VOLATILE SILICONE OIL, AND PROCESS USING THE SAME

(71) Applicant: L'OREAL, Paris (FR)

(72) Inventors: Roshanak DEBEAUD, Chevilly La Rue (FR); Sandrine FERNANDES, Chevilly La Rue (FR); Roberto CAVAZZUTI, Chevilly La Rue (FR)

(73) Assignee: L'OREAL, Paris (FR)

(21) Appl. No.: 16/470,848

(22) PCT Filed: Dec. 21, 2017
(86) PCT No.: PCT/EP2017/084024

§ 371 Date: Jun. 18, 2019

(30) Foreign Application Priority Data
Dec. 21, 2016 (FR) 1662995

(51) Int. Cl. A61K 8/89; A61K 8/92; A61K 8/06; A61Q 1/02

(52) U.S. Cl. CPC A61K 8/89; A61Q 1/02; A61K 8/062; A61K 8/92

(57) ABSTRACT

The present invention relates to a cosmetic composition comprising: at least 10% by weight, relative to the weight of the composition, of water; •at least 10% by weight, relative to the weight of the composition, of at least one silicone resin; •at least one non-volatile silicone oil, not comprising any nitrogen atoms, or any —Si—H groups, or any (poly)oxyalkylene groups, the oxyalkylene unit being C₂-C₃, or any (poly)glycerol groups; •optionally at least one first non-volatile hydrocarbon-based oil in a content of less than 15% by weight, relative to the weight of the composition; said non-volatile hydrocarbon-based oil(s) being chosen from: —C₁₀-C₂₆ alcohols, preferably monoalcohols; —optionally hydroxylated monoesters, diesters or triesters of a C₂-C₈ monocarboxylic or polycarboxylic acid and of a C₂-C₈ alcohol, which are optionally hydroxylated; —esters of a C₂-C₈ polyol and of one or more C₂-C₈ carboxylic acids; •optionally, at least one volatile oil in a content such that the silicone resin/volatile oil(s) weight ratio is greater than or equal to 1. The invention also relates to a process for making up and/or caring in particular for the lips, the skin, more particularly the cheeks, in which the abovementioned composition is applied.

The present invention relates to a composition, intended in particular for making up and/or caring for the lips and the skin, comprising at least 10% by weight of water, at least 10% by weight of a silicone resin, at least one non-volatile silicone oil and optionally additional particular hydrocarbon-based oils, and also to a process for treating and/or making up keratin materials, in particular the lips.

The development of compositions for making up and/or caring for the lips, in particular fluid compositions such as liquid lipsticks, which are stable and endowed with satisfactory properties in terms of application (glidance on application, ease of spreading and fineness of the deposit), but also in terms of the makeup effect of the deposit on the lips, for instance the absence of migration of the deposit, preferably without becoming tacky, is an ongoing objective.

Generally, formulations corresponding to liquid presentation forms conventionally comprise oils, which in particular provide gloss, optionally waxes for structuring the compositions, fillers especially for thickening the composition, film-forming polymers, and dyestuffs.

These liquid lipsticks must be sufficiently fluid to be easily applied, but not too fluid, so as not to degrade the stability of the composition (pigment sedimentation) and the ease of application (running).

With conventional liquid lipstick compositions, it is noted that the deposit is relatively thick, thereby giving it a more or less tacky nature, especially induced by the use of these oils and of the polymers present. This nature may be reflected especially by adhesion of the made-up lips to each other, causing the user an unpleasant sensation in terms of comfort.

Liquid lipstick compositions have quite recently appeared on the market, in the form of direct or inverse aqueous emulsions. When they are applied, they provide a fresh effect, and are sparingly tacky or non-tacky, and comfortable, and remain so once deposited. They also make it possible to obtain a homogeneous, sufficiently glossy deposit which has satisfactory persistence and transfer resistance.

Nevertheless, it is desired to further improve the persistence of these compositions, and also their transfer resistance, without, however, losing any gloss on application or any comfort. Specifically, it is not uncommon to find that such compositions have the drawback of leaving either a matt deposit or a deposit which is more uncomfortable since it is very present on the lips (with an impression of reduction of the mobility of the lips), often accompanied by a sensation of dryness.

It is also advantageous to have available compositions that can be applied both to the lips and to the skin, in particular to the face and more precisely the cheeks.

Compositions, in particular liquid compositions, which can produce a deposit whose persistence and transfer resistance are improved without any loss of comfort are thus sought. Compositions of which the deposit on the lips or the skin, in particular the cheeks, although very thin, makes it possible to obtain a visible, glossy colour, with improved colour persistence, are also sought. These aims and others are achieved by the present invention, one subject of which is a cosmetic composition comprising:
at least 10% by weight, relative to the weight of the composition, of water;
at least 10% by weight, relative to the weight of the composition, of at least one silicone resin;
at least one non-volatile silicone oil, not comprising any nitrogen atoms, or any —Si—H groups, or any (poly)oxyalkylene groups, the oxyalkylene unit being C₂-C₃, or any (poly)glycerol groups and preferably not comprising any C₈-C₂₂ alkyl groups when the non-volatile silicone oil does not comprise any phenyl groups;
optionally at least one first non-volatile hydrocarbon-based oil in a content of less than 15% by weight, relative to the weight of the composition; said non-volatile hydrocarbon-based oil(s) being chosen from:
   C₁₀-C₂₆ alcohols, preferably branched monoalcohols;
   optionally hydroxylated monoesters, diesters or triesters of a C₂-C₈ monocarboxylic or polycarboxylic acid and of a C₂-C₈ alcohol, which are optionally hydroxylated;
   esters of a C₂-C₈ polyol and of one or more C₂-C₈ carboxylic acids;
optionally, at least one volatile oil in a content such that the silicone resin/volatile oil(s) weight ratio is greater than or equal to 1.

The film deposited on the lips has the advantage of being thin and affords a fresh effect on application, without significant migration into the wrinkles and fine lines.

The composition is stable, easy to apply, comfortable on application since the deposit obtained is sparingly tacky or non-tacky, and remains so once in place. Moreover, the deposit does not give an impression of dryness of the lips.

In addition, the deposit obtained by applying the composition according to the invention has satisfactory gloss.

The colour persistence of the deposit obtained by applying the composition is improved, as is its transfer resistance.

It should be noted that, in the remainder of the description, unless otherwise indicated, the limits indicated for a range are included in that range.

The expressions “at least one” and “several” are used without distinction.

The composition according to the invention is advantageously in a liquid form.

The term “liquid” means a fluid texture, the viscosity of which at 25° C. is more particularly between 0.05 and 10 Pa·s, preferably between 0.1 and 8 Pa·s.

Protocol for Measuring the Viscosity:

The viscosity measurement is generally performed at 25° C., using a Rheomat RM 100 viscometer equipped with a no. 2 or 3 spindle, the measurement being performed after 10 minutes of rotation of the spindle in the composition, at a shear rate of 200 revolutions/min (rpm).

Protocol for Measuring the Stability:

According to the invention, a composition is stable when substantially no phase separation is observed, or any sedimentation of the solid particles if the composition comprises any, when the following protocol is performed:

Centrifugations are performed at room temperature on samples of composition introduced into sterile, 15-ml graduated plastic centrifugation tubes made of propylene, with a conical end and a stopper (ref Biologix 10-9152) to be filled with 8.6 g of composition).

The measurement is more particularly performed 24 hours after making the composition.

Centrifugation of the compositions according to the invention for 10 minutes at 450×g (expressed in acceleration units) does not reveal any instability (no phase separation).

Advantageously, after a step of centrifugation for 1 hour at 900×g (expressed in acceleration units), no phase separation of the compositions thus treated is observed, or, if phase separation appears, the latter reveals a phase either a pellet in the tube, which is less than or equal to 5 mm, or a supernatant in the top of the tube, which is less than or equal to 2 mm.

According to a particularly advantageous embodiment of the invention, the composition is in the form of a composition whose aqueous phase is the continuous phase and whose oily phase is the dispersed phase (oil-in-water direct emulsion).

The term “composition with an aqueous continuous phase” more particularly means that a pH value can be measured for the composition with a suitable electrode (for example an MPC227 conductimeter from Mettler Toledo).

### Silicone Resin

As indicated previously, the composition according to the invention comprises at least one silicone resin.

More generally, the term “resin” means a compound whose structure is three-dimensional. Thus, for the purposes of the present invention, a polydimethylsiloxane is not a silicone resin.

The nomenclature of silicone resins (also known as siloxane resins) is known under the name “MDTQ”, the resin being described as a function of the various siloxane monomer units it comprises, each of the letters “MDTQ” characterizing a type of unit.

The letter M represents the monofunctional unit of formula R1R2R3SiO_{1/2}, the silicon atom being bonded to only one oxygen atom in the polymer comprising this unit.

The letter D means a difunctional unit R1R2SiO_{2/2} in which the silicon atom is bonded to two oxygen atoms.

The letter T represents a trifunctional unit of formula R1SiO_{3/2}.

Such resins are described, for example, in the Encyclopedia of Polymer Science and Engineering, vol. 15, John Wiley and Sons, New York, (1989), pp. 265-270, and U.S. Pat. Nos. 2,676,182, 3,627,851, 3,772,247, 5,248,739 or else U.S. Pat. Nos. 5,082,706, 5,319,040, 5,302,685 and 4,935,484.

In the units M, D and T defined previously, Ri, namely R₁, R₂ and R₃, which may be identical or different, represent a hydrocarbon-based radical (especially alkyl) containing from 1 to 10 carbon atoms, a phenyl group, a phenylalkyl group or else a hydroxyl group.

Finally, the letter Q means a tetrafunctional unit SiO_{4/2} in which the silicon atom is bonded to four oxygen atoms, which are themselves bonded to the rest of the polymer.

Various silicone resins with different properties may be obtained from these different units, the properties of these polymers varying as a function of the type of monomer (or unit), the nature and number of the radical(s) Ri, the length of the polymer chain, the degree of branching and the size of the side chains.

As silicone resins that may be used in the compositions according to the invention, use may be made, for example, of silicone resins of MQ type, of T type or of MQT type.

### MQ Resins:

As examples of silicone resins of MQ type, mention may be made of the alkyl siloxysilicates of formula [(R1)₃SiO_{1/2}]ₓ(SiO_{4/2})_{y} (units MQ) in which x and y are integers ranging from 50 to 80, and such that the group R1 represents a radical as defined previously, and is preferably an alkyl group containing from 1 to 8 carbon atoms or a hydroxyl group, preferably a methyl group.

As examples of solid silicone resins of MQ type of trimethyl siloxysilicate type, mention may be made of those sold under the reference SR1000 by the company Momentive Performance Materials, under the reference MQ 1600 by Dow Corning or under the reference Belsil TMS 803 by the company Wacker.

As silicone resins comprising MQ siloxysilicate units, mention may also be made of phenylalkyl siloxysilicate resins, such as phenylpropyldimethyl siloxysilicate (Silshine 151 sold by the company Momentive Performance Materials). The preparation of such resins is described especially in U.S. Pat. No. 5,817,302.

### T Resins:

Examples of silicone resins of T type that may be mentioned include the polysilsesquioxanes of formula (RSiO_{3/2})ₓ (units T) in which x is greater than 100 and such that the group R is an alkyl group containing from 1 to 10 carbon atoms, said polysilsesquioxanes also possibly comprising Si—OH end groups.

Mention may also be made of polymethylsilsesquioxanes, which are polysilsesquioxanes in which none of the methyl radicals is substituted with another group. Such polymethylsilsesquioxanes are described, for example, in U.S. Pat. No. 5,246,694.

Polymethylsilsesquioxane resins that may preferably be used are those in which R represents a methyl group, for instance those sold:
by the company Wacker under the reference Resin MK, such as Belsil PMS MK: polymer comprising CH₃SiO_{3/2} repeating units (units T), which may also comprise up to 1% by weight of (CH₃)₂SiO_{2/2} units (units D) and having an average molecular weight of about 10 000 g/mol, or
by the company Shin-Etsu under the references KR-220L, which are composed of units T of formula CH₃SiO_{3/2} and contain Si—OH (silanol) end groups, under the reference KR-242A, which comprise 98% of units T and 2% of dimethyl units D and contain Si—OH end groups, or else under the reference KR-251, comprising 88% of units T and 12% of dimethyl units D and contain Si—OH end groups.
by the company Dow Corning under the references Dow Corning 670 Fluid, Dow Corning 680 Fluid, as a mixture in cyclopentasiloxane and in isododecane, respectively.

### MQT Resins:

Resins comprising MQT units that are especially known are those mentioned in U.S. Pat. No. 5,110,890.

A preferred form of resins of MQT type are MQT-propyl (also known as MQTpr) resins. Such resins that may be used in the compositions according to the invention are especially the resins described and prepared in patent application WO 2005/075 542, the content of which is incorporated herein by reference.

The MQ-T-propyl resin preferably comprises the following units:

(i) (R1₃SiO_{1/2})ₐ

(ii) (R2₂SiO_{2/2})_{b}

(iii) (R3SiO_{3/2})_{c} and

(iv) (SiO_{4/2})_{d}

with
R1, R2 and R3 independently representing a hydrocarbon-based radical (especially alkyl) containing from 1 to 10 carbon atoms, a phenyl group, a phenylalkyl group or a hydroxyl group and preferably an alkyl radical containing from 1 to 8 carbon atoms or a phenyl group,
a, b, c and d being mole fractions,
a being between 0.05 and 0.5,
b being between zero and 0.3,
c being greater than zero,
d being between 0.05 and 0.6,
a+b+c+d=1,
on condition that more than 40 mol % of the groups R3 of the siloxane resin are propyl groups.

Preferably, the siloxane resin comprises the following units:

(i) (R1₃SiO_{1/2})ₐ

(iii) (R3SiO_{3/2})_{c} and

(iv) (SiO_{4/2})_{d}

with
R₁ and R₃ independently representing an alkyl group containing from 1 to 8 carbon atoms, R₁ preferably being a methyl group and R3 preferably being a propyl group,
a being between 0.05 and 0.5 and preferably between 0.15 and 0.4,
c being greater than zero, preferably between 0.15 and 0.4,
d being between 0.05 and 0.6, preferably between 0.2 and 0.6 or alternatively between 0.2 and 0.55,

a+b+c+d=1, and a, b, c and d being mole fractions,
on condition that more than 40 mol % of the groups R₃ of the siloxane resin are propyl groups.

The siloxane resins that may be used according to the invention may be obtained via a process comprising the reaction of:

A) an MQ resin comprising at least 80 mol % of units (R1₃SiO_{1/2})ₐ and (SiO_{4/2})_{d},
R1 representing an alkyl group containing from 1 to 8 carbon atoms, an aryl group, a carbinol group or an amino group,
a and d being greater than zero,
the ratio a/d being between 0.5 and 1.5,

and

B) a T-propyl resin comprising at least 80 mol % of units (R3SiO_{3/2})_{c},
R3 representing an alkyl group containing from 1 to 8 carbon atoms, an aryl group, a carbinol group or an amino group,
c being greater than zero,
on condition that at least 40 mol % of the groups R3 are propyl groups,
in which the mass ratio A/B is between 95/5 and 15/85 and preferably the mass ratio A/B is 30/70.

Advantageously, the mass ratio A/B is between 95/5 and 15/85. Preferably, the ratio A/B is less than or equal to 70/30. These preferred ratios have proven to afford comfortable deposits.

Preferably, the composition according to the invention comprises, as silicone resin, at least one resin of MQ type as described previously.

In particular, the silicone resin is a siloxysilicate resin, preferably a trimethylsiloxysilicate resin.

Advantageously, the silicone resin is present in a content of at least 10% by weight relative to the weight of the composition. Preferably, the content of silicone resin represents between 10 and 45% by weight, preferably between 12 and 40% by weight, in particular between 15 and 35% by weight, or even from 18 to 35% by weight, relative to the weight of the composition.

The silicone resin may be used in powder form, in a form dissolved in a solvent, in a form conveyed in a liquid or in a form emulsified in water. It should be noted that, in the latter case, the silicone resin is preferably in a conveyed form, advantageously dissolved in a solvent, and then emulsified. Preferably the silicon resin is used in a form conveyed in a solvent, or in a form emulsified in water.

As regards the silicone resins conveyed in a solvent, said solvent is usually chosen from volatile or non-volatile, apolar hydrocarbon-based oils and silicone oils, preferably volatile oils.

The term “volatile oil” means an oil with a non-zero vapour pressure, at room temperature (25° C.) and atmospheric pressure, ranging in particular from 0.13 Pa to 40 000 Pa, in particular ranging up to 13 000 Pa and more particularly ranging up to 1300 Pa. For example, the vapor pressure can be measured by the static method or the isothermal thermogravimetry effusion method, depending on the vapor pressure (OECD 104 standard).

Volatile hydrocarbon-based oils that may especially be mentioned include alkanes, preferably branched alkanes of 8 to 16 carbon atoms, especially such as C₈-C₁₆ isoalkanes (also known as isoparaffins), isododecane, isodecane and isohexadecane.

Volatile silicone oils that may be mentioned include linear or cyclic silicone oils, such as linear or cyclic polydimethylsiloxanes (PDMSs) containing from 3 to 7 silicon atoms.

Examples of such oils that may be mentioned include octyl trimethicone, hexyl trimethicone, decamethylcyclopentasiloxane, octamethylcyclotetrasiloxane, dodecamethylcyclohexasiloxane, decamethyltetrasiloxane, methyl trimethicone, polydimethylsiloxanes such as those sold under the reference DC 200 by Dow Corning or KF 96 A from Shin-Etsu; alone or as mixtures.

Among the silicone resins, especially of MQ type, which are in a form conveyed in a solvent, mention may be made especially of Koboguard® MQ65TMF (mixture of trimethylsiloxysilicate and methyl trimethicone) sold by Kobo; KF-7312J (mixture in cyclopentasiloxane), KF-7312K, KF-7312L (mixtures in dimethicone), KF-7312T (mixture in trimethicone), X-21-5249 (mixture in cyclopentasiloxane), X-21-5249L (mixture in dimethicone), X-21-5250, X-21-5250L (mixture in cyclopentasiloxane and dimethicone, respectively), X-21-5595, X-21-5616 (mixtures in isododecane), KF-9021, KF-9021L (mixtures in cyclopentasiloxane and in dimethicone, respectively), sold by Shin-Etsu; Silsoft 74, Silshine 151 (mixtures in isododecane) from Momentive Performance Materials; Xiameter RSN-0749 Resin, Dow Corning 749 Fluid (mixtures in cyclopentasiloxane), Dow Corning 593 Fluid (mixture in dimethicone) from Dow Corning.

As regards the silicone resins that are in the form of emulsions in water, mention may be made, for example, of KM-9717 (emulsion in the presence of an anionic surfactant, comprising a low-viscosity silicone), X-52-8005 (emulsion in the presence of a nonionic surfactant, comprising a low-viscosity silicone), sold by Shin-Etsu.

### Non-Volatile Silicone Oils

The composition according to the invention moreover comprises at least one non-volatile silicone oil, not comprising any nitrogen atoms, or any —Si—H groups, or any (poly)oxyalkylene groups, the oxyalkylene unit of which is C₂-C₃, or any (poly)glycerol groups and preferably not comprising any C₈-C₂₂ alkyl groups when the non-volatile silicone oil does not comprise any phenyl groups.

The term “silicone oil” means an oil containing at least one silicon atom, and especially containing Si—O groups.

In addition, the term “oil” denotes a compound that is liquid at 25° C. and atmospheric pressure (1.013×10⁵ Pa).

The term “non-volatile oil” means an oil whose vapour pressure at 25° C. and atmospheric pressure is non-zero and is less than 10⁻³ mmHg (0.13 Pa).

Preferably, the non-volatile silicone oil is nonionic.

According to the invention, the term “nonionic” means that the compound does not comprise any ionic groups, irrespective of the pH of the composition.

Among the non-volatile silicone oils that may be used in the present invention, examples that may be mentioned include non-volatile non-phenyl silicone oils and non-volatile phenyl silicone oils.

The silicone oil may be used in unmodified form, or in a form dissolved in at least one volatile or non-volatile oil, or in the form of an emulsion.

### Non-Volatile Non-Phenyl Silicone Oils

The expression “non-phenyl silicone oil” denotes a silicone oil not comprising phenyl substituents.

Representative examples of these non-volatile non-phenyl silicone oils that may be mentioned include polydimethylsiloxanes; vinyl dimethicones and copolymers with dimethicone.

Moreover, the term “dimethicone” (INCI name) corresponds to a polydimethylsiloxane (chemical name).

In particular, these oils may be chosen from the following non-volatile oils:
polydimethylsiloxanes (PDMSs),
polydimethylsiloxanes comprising functional groups such as hydroxyl groups,
divinyl dimethicone/dimethicone copolymers,
mixtures thereof.

The non-volatile non-phenyl silicone oil may be chosen in particular from silicones of formula (I):

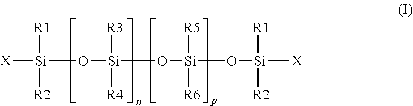

in which:
R₁, R₂, R₅ and R₆ are, together or separately, an alkyl radical containing from 1 to 6 carbon atoms,
R₃ and R₄ are, together or separately, an alkyl radical containing 1 to 6 carbon atoms, or a hydroxyl radical,
X is an alkyl radical containing from 1 to 6 carbon atoms, a hydroxyl radical,
n and p are integers chosen so as to have a fluid compound, in particular of which the viscosity at 25° C. is between 8 centistokes (cSt) (8×10−⁶ m²/s) and 800 000 cSt, advantageously less than 600 000 cSt.

As non-volatile non-phenyl silicone oils that are suitable for performing the invention, mention may be made of those for which:
the substituents R₁ to R₆ and X represent a methyl group, and p and n are such that the viscosity is 500 000 cSt, for example the product sold under the name Silsoft SE30 by the company Momentive Performance Materials, the product sold under the name AK 500000 by the company Wacker, the product sold under the name Mirasil DM 500 000 by the company Bluestar, and the product sold under the name Dow Corning 200 Fluid 500 000 cSt by the company Dow Corning,
the substituents R₁ to R₆ and X represent a methyl group, and p and n are such that the viscosity is 60 000 cSt, for example the product sold under the name Dow Corning 200 Fluid 60 000 CS by the company Dow Corning, and the product sold under the name Belsil DM 60 000 by the company Wacker,
the substituents R₁ to R₆ and X represent a methyl group, and p and n are such that the viscosity is 100 cSt or 350 cSt, for example the products sold respectively under the names Belsil DM100 and Dow Corning 200 Fluid 350 CS by the company Dow Corning, and
the substituents R₁ to R₆ represent a methyl group, the group X represents a hydroxyl group, and n and p are such that the viscosity is 700 cSt, for example the product sold under the name Baysilone Fluid T0.7 by the company Momentive Performance Materials.

Dimethiconols such as the products Xiameter PMX-1503 (as a mixture with a dimethicone) and Xiameter PMX-1502 (mixture with C11-13 isoparaffin, isohexadecane, dimethicone) from Dow Corning may also be suitable for use.

The silicone oil may be used in unmodified form, or in a form dissolved in at least one volatile or non-volatile oil, or in the form of an emulsion.

As examples of emulsions of non-volatile silicone oil, mention may be made of the products Xiameter MEM-1352 Emulsion (dimethicone, laureth-23, C12-15 pareth-3), Xiameter MEM 1491 Emulsion (dimethicone, laureth-23, C12-15 pareth-3), Xiameter MEM-1691 Emulsion (dimethicone, C12-13 pareth-4 and C12-13 pareth-23 and salicylic acid), Xiameter MEM 1652 Emulsion (dimethicone, C12-13 pareth-23; C12-C15 pareth 3, salicylic acid), Xiameter MEM 1664 Emulsion (dimethicone, laureth-4, laureth-23), Xiameter MEM-2664 Emulsion (dimethicone, laureth-23, laureth-4), Xiameter MEM-1784 Emulsion or Dow Corning CE2060 (dimethicone, cocamidopropylbetaine, C12-15 pareth-3, guar hydroxypropyltrimonium chloride), Xiameter MEM-1785 Emulsion, Xiameter MEM-1784 Emulsion or Xiameter MEM-1788 Emulsion (dimethiconol, TEA dodecylbenzenesulfonate), Belsil DM 3560 VP (dimethiconol, sodium dodecylbenzenesulfonate, trideceth-10), Dow Corning HMW 2220 nonionic emulsion (divinyl dimethicone/dimethicone copolymer, C12-13 pareth-3, C12-13 pareth-23), sold by Dow Corning; KM-740T (nonionic); KM-860A (nonionic), KM-9736A (anionic), KM-9737A (anionic), KM-9738A (anionic), KM-862T (nonionic), KM-752T (anionic), KM-9774 (anionic), from Shin-Etsu.

### Non-Volatile Phenyl Silicone Oils

The expression “phenyl silicone oil” denotes a silicone oil bearing at least one phenyl substituent.

These non-volatile phenyl silicone oils may be chosen from those also bearing at least one dimethicone fragment, or from those not bearing any. It should be noted that the term “dimethicone fragment” denotes a divalent siloxane group in which the silicon atom bears two methyl radicals, this group not being located at the end of the molecule. It can be represented by the following formula: —(Si(CH₃)₂—O)—.

The non-volatile phenyl silicone oil may thus be chosen from:
phenyl silicone oils optionally bearing a dimethicone fragment corresponding to formula (I) below:

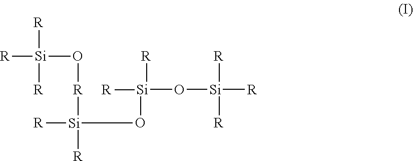

in which the groups R, which are monovalent or divalent, represent, independently of each other, a methyl, methylene, phenyl or phenylene, with the proviso that at least one group R represents a phenyl.

Preferably, in this formula, the phenyl silicone oil comprises at least three phenyl groups, for example at least four, at least five or at least six.
phenyl silicone oils optionally bearing a dimethicone fragment corresponding to formula (II) below:

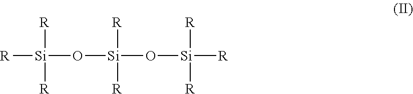

in which the groups R represent, independently of each other, a methyl or a phenyl, with the proviso that at least one group R represents a phenyl.

Preferably, in this formula, the compound of formula (II) comprises at least three phenyl groups, for example at least four or at least five.

Mixtures of different phenylorganopolysiloxane compounds described previously may be used.

Examples that may be mentioned include mixtures of triphenyl-, tetraphenyl- or pentaphenylorganopolysiloxanes.

Among the compounds of formula (II), mention may be made more particularly of phenyl silicone oils not bearing any dimethicone fragments, corresponding to formula (II) in which at least 4 or at least 5 radicals R represent a phenyl radical, the remaining radicals representing methyls.

Such non-volatile phenyl silicone oils are preferably trimethylpentaphenyltrisiloxane or tetramethyltetraphenyltrisiloxane. They are in particular sold by Dow Corning under the reference PH-1555 HRI or Dow Corning 555 Cosmetic Fluid (chemical name: 1,3,5-trimethyl-1,1,3,5,5-pentaphenyltrisiloxane; INCI name: trimethylpentaphenyltrisiloxane), or the tetramethyltetraphenyltrisiloxane sold under the reference Dow Corning 554 Cosmetic Fluid by Dow Corning may also be used.

They correspond especially to formulae (III) and (III′) below:

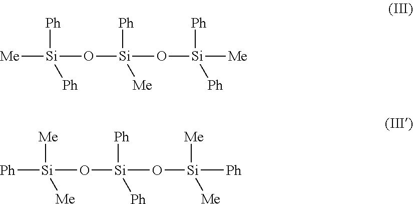

in which Me represents methyl, and Ph represents phenyl.
phenyl silicone oils bearing at least one dimethicone fragment corresponding to formula (IV) below:

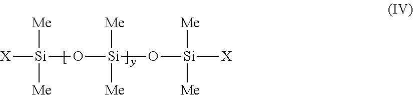

in which Me represents methyl, y is between 1 and 1000 and X represents —CH₂—CH(CH₃)(Ph).
phenyl silicone oils optionally bearing a dimethicone fragment, corresponding to formula (V) below, and mixtures thereof:

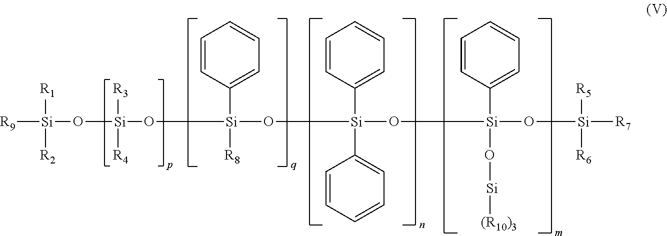

in which:
R₁ to R₁₀, independently of each other, are saturated or unsaturated and linear, cyclic or branched C₁-C₃₀ hydrocarbon-based radicals,
m, n, p and q are, independently of each other, integers between 0 and 1000, preferably 900, with the proviso that the sum m+n+q is other than 0.

Preferably, the sum m+n+q is between 1 and 100. Advantageously, the sum m+n+p+q is between 1 and 1000, more particularly between 1 and 900 and preferably between 1 and 800.

Preferably, q is equal to 0.

More particularly, R₁ to R₁₀ represent, independently of each other, a saturated or unsaturated, preferably saturated, and linear or branched C₁-C₃₀ hydrocarbon-based radical, and in particular a preferably saturated C₁-C₂₀, in particular C₁-C₁₈, hydrocarbon-based radical, or a monocyclic or polycyclic C₆-C₁₄ and in particular C₁₀-C₁₃ aryl radical, or an aralkyl radical, the alkyl part of which is preferably a C₁-C₃ alkyl.

Preferably, R₁ to R₁₀ may each represent a methyl, ethyl, propyl, butyl, isopropyl, decyl, dodecyl or octadecyl radical, or as a variant a phenyl, tolyl, benzyl or phenethyl radical. R₁ to R₁₀ may in particular be identical, and in addition may be a methyl radical.

As particular embodiments of formula (V), mention may be made of:
phenyl silicone oils optionally bearing at least one dimethicone fragment corresponding to formula (VI) below, and mixtures thereof:

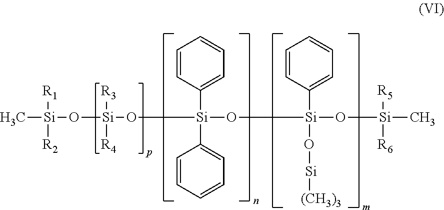

in which:
R₁ to R₆, independently of each other, are saturated or unsaturated, linear, cyclic or branched C₁-C₃₀ hydrocarbon-based radicals, a preferably C₆-C₁₄ aryl radical or an aralkyl radical, the alkyl part of which is a C₁-C₃ alkyl,
m, n and p are, independently of each other, integers between 0 and 1000, preferably between 0 and 100, with the proviso that the sum n+m is between 1 and 1000, preferably between 1 and 100.

Preferably, R₁ to R₆ represent, independently of each other, a C₁-C₂₀, in particular C₁-C₁₈, preferably alkyl, hydrocarbon-based radical, or a C₆-C₁₄ aryl radical which is monocyclic (preferably a C₆ aryl radical) or polycyclic and in particular a C₁₀-C₁₃ aryl radical, or an aralkyl radical (preferably the aryl part is a C₆ aryl; the alkyl part is a C₁-C₃ alkyl).

Preferably, R₁ to R₆ may each represent a methyl, ethyl, propyl, butyl, isopropyl, decyl, dodecyl or octadecyl radical, or as a variant a phenyl, tolyl, benzyl or phenethyl radical.

R₁ to R₆ may in particular be identical, and in addition may be a methyl radical. Preferably, m=1 or 2 or 3, and/or n=0 and/or p=0 or 1 may be applied, in formula (VI).

According to a particular embodiment, the non-volatile phenyl silicone oil is chosen from phenyl silicone oils bearing at least one dimethicone fragment.

Preferably, such oils correspond to compounds of formula (VI) in which:
m=0 and n and p are, independently of each other, integers between 1 and 100.

Preferably, R₁ to R₆ are methyl radicals.

According to this embodiment, the silicone oil is preferably chosen from a diphenyl dimethicone, such as KF-54 from Shin-Etsu (400 cSt), KF54HV from Shin-Etsu (5000 cSt), KF-50-300CS from Shin-Etsu (300 cSt), KF-53 from Shin-Etsu (175 cSt) or KF-50-100CS from Shin-Etsu (100 cSt).
p is between 1 and 1000, the sum n+m is between 1 and 1000, and n=0.

These phenyl silicone oils optionally bearing at least one dimethicone fragment correspond more particularly to formula (VII) below:

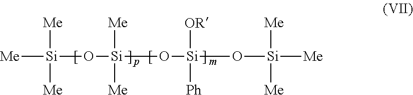

in which Me is methyl and Ph is phenyl, OR′ represents an —OSiMe₃ group and p is 0 or is between 1 and 1000, and m is between 1 and 1000. In particular, m and p are such that compound (VII) is a non-volatile oil.

According to a first embodiment of a non-volatile phenyl silicone bearing at least one dimethicone fragment, p is between 1 and 1000 and m is more particularly such that compound (VII) is a non-volatile oil. Use may be made, for example, of trimethylsiloxyphenyl dimethicone, sold in particular under the reference Belsil PDM 1000 or Belsil PDM 20 by the company Wacker.

According to a second embodiment of a non-volatile phenyl silicone not bearing any dimethicone fragments, p is equal to 0 and m is between 1 and 1000, and in particular is such that compound (VII) is a non-volatile oil.

Use may be made, for example, of phenyl trimethicone, sold in particular under the reference Dow Corning 556 Cosmetic Grade Fluid (DC556).
non-volatile phenyl silicone oils not bearing any dimethicone fragments corresponding to formula (VIII) below, and mixtures thereof:

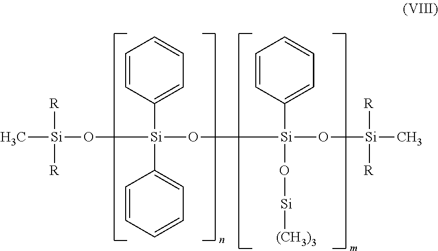

in which:
R, independently of each other, are saturated or unsaturated, linear, cyclic or branched C₁-C₃₀ hydrocarbon-based radicals, preferably R is a C₁-C₃₀ alkyl radical, a preferably C₆-C₁₄ aryl radical, or an aralkyl radical, the alkyl part of which is a C₁-C₃ alkyl,
m and n are, independently of each other, integers between 0 and 100, with the proviso that the sum n+m is between 1 and 100.

Preferably, R, independently of each other, represent a saturated or unsaturated, preferably saturated, linear or branched C₁-C₃₀ hydrocarbon-based radical, and in particular a preferably saturated, C₁-C₂₀, in particular C₁-C₁₈ and more particularly C₄-C₁₀, hydrocarbon-based radical, a monocyclic or polycyclic C₆-C₁₄, and in particular C₁₀-C₁₃, aryl radical, or an aralkyl radical of which preferably the aryl part is a C₆ aryl and the alkyl part is a C₁-C₃ alkyl.

Preferably, the groups R may each represent a methyl, ethyl, propyl, butyl, isopropyl, decyl, dodecyl or octadecyl radical, or as a variant a phenyl, tolyl, benzyl or phenethyl radical.

The groups R may in particular be identical, and in addition may be a methyl radical.

According to a preferred embodiment, n is an integer between 0 and 100 and m is an integer between 1 and 100, with the proviso that the sum n+m is between 1 and 100, in formula (VIII). Preferably, R is a methyl radical.

According to one embodiment, a phenyl silicone oil of formula (VIII) with a viscosity at 25° C. of between 5 and 1500 mm²/s (i.e. 5 to 1500 cSt), and preferably with a viscosity of between 5 and 1000 mm²/s (i.e. 5 to 1000 cSt), may be used.

According to this embodiment, the non-volatile phenyl silicone oil is preferably chosen from phenyl trimethicones (when n=0, m more particularly between 1 and 3) such as Dow Corning 556 Cosmetic Grade Fluid from Dow Corning (20 cSt), or else from diphenylsiloxyphenyl trimethicone oil (when m and n are between 1 and 100) such as KF56 A from Shin-Etsu, or the oil Silbione 70663V30 from Rhône-Poulenc (28 cSt). The values in parentheses represent the viscosities at 25° C.
phenyl silicone oils optionally bearing at least one dimethicone fragment corresponding to the following formula, and mixtures thereof:

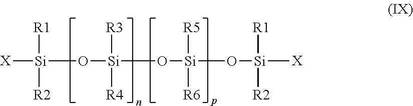

in which:

R₁, R₂, R₅ and R₆, which may be identical or different, are an alkyl radical containing 1 to 6 carbon atoms,

R₃ and R₄, which may be identical or different, are an alkyl radical containing from 1 to 6 carbon atoms or an aryl radical (preferably C₆-C₁₄), with the proviso that at least one from among R₃ and R₄ is a phenyl radical,

X is an alkyl radical containing from 1 to 6 carbon atoms, a hydroxyl radical or a vinyl radical,

n and p being an integer greater than or equal to 1, chosen so as to give the oil a weight-average molecular mass preferably less than 150 000 g/mol and more preferably less than 100 000 g/mol.
and a mixture thereof.

More particularly, the composition comprises at least one non-volatile silicone oil chosen from polydimethylsiloxanes; non-volatile phenyl silicone oils bearing a dimethicone fragment, and also mixtures thereof.

Preferably, the composition comprises as non-volatile silicone oil(s), polydimethylsiloxanes and phenyl silicones of formula (V), and also mixtures thereof.

Preferably, the non-volatile phenyl silicone oil(s) bearing a dimethicone fragment are chosen from trimethylsiloxyphenyl dimethicones and diphenyl dimethicones, and also mixtures thereof.

Needless to say, the composition according to the invention may also comprise, in addition to the abovementioned non-volatile phenyl silicone oils, non-volatile phenyl silicone oils not bearing any dimethicone fragments, for instance phenyl trimethicones, trimethylpentaphenyltrisiloxanes and tetramethyltetraphenyltrisiloxanes, alone or as mixtures.

Advantageously, the composition has a content of non-volatile silicone oil(s) ranging from 2% to 35% by weight and preferably from 8% to 30% by weight relative to the weight of the composition.

### Non-Volatile Hydrocarbon-Based Oils

The composition according to the invention may optionally comprise at least one particular non-volatile hydrocarbon-based oil.

The term “oil” denotes a compound that is liquid at 25° C. and atmospheric pressure (1.013×10⁵ Pa).

### First Non-Volatile Hydrocarbon-Based Oils

In particular, if the first oil is chosen from the following oils:
C₁₀-C₂₆ alcohols, preferably monoalcohols;
optionally hydroxylated monoesters, diesters or triesters of a C₂-C₈ monocarboxylic or polycarboxylic acid and of a C₂-C₈ alcohol, which are optionally hydroxylated;
esters of a C₂-C₈ polyol and of one or more C₂-C₈ carboxylic acids;

then their content in the composition is less than 15% by weight, more particularly between 0.5% and less than 15% by weight, relative to the weight of the composition.

Preferably, the content of this type of oil is less than 10% by weight, even more particularly less than 5% by weight and even more preferentially less than 2% by weight, relative to the weight of the composition.

In accordance with an even more preferred embodiment of the invention, the composition according to the invention does not comprise any additional first oil.

More particularly, these oils are chosen from:
C₁₀-C₂₆ alcohols, preferably monoalcohols;

More particularly, the C₁₀-C₂₆ alcohols are saturated or unsaturated, and branched or unbranched, and comprise from 10 to 26 carbon atoms.

Advantageously, the C₁₀-C₂₆ alcohols are fatty alcohols, which are preferably branched when they comprise at least 16 carbon atoms.

As examples of fatty alcohols that may be used according to the invention, mention may be made of linear or branched fatty alcohols, of synthetic origin or alternatively of natural origin, for instance alcohols derived from plant materials (coconut, palm kernel, palm, etc.) or animal materials (tallow, etc.).

Use may also be made of other long-chain alcohols, for instance ether alcohols or “Guerbet” alcohols.

Finally, use may also be made of certain more or less long fractions of alcohols of natural origin, for instance coconut (C₁₂ to C₁₆) or tallow (C₁₆ to C₁₈).

Preferably, the fatty alcohol comprises from 10 to 24 carbon atoms and more preferentially from 12 to 22 carbon atoms.

As particular examples of fatty alcohols that may preferably be used, mention may be made especially of lauryl alcohol, isostearyl alcohol, oleyl alcohol, 2-butyloctanol, 2-undecylpentadecanol, 2-hexyldecyl alcohol, isocetyl alcohol and octyldodecanol, and mixtures thereof.

According to an advantageous embodiment of the invention, the alcohol is chosen from octyldodecanol.
optionally hydroxylated monoesters, diesters or triesters of a C₂-C₈ monocarboxylic or polycarboxylic acid and of a C₂-C₈ alcohol.

In particular:
optionally hydroxylated monoesters of a C₂-C₈ carboxylic acid and of a C₂-C₈ alcohol,
optionally hydroxylated diesters of a C₂-C₈ dicarboxylic acid and of a C₂-C₈ alcohol, such as diisopropyl adipate, 2-diethylhexyl adipate, dibutyl adipate, diisostearyl adipate or 2-diethylhexyl succinate,
optionally hydroxylated triesters of a C₂-C₈ tricarboxylic acid and of a C₂-C₈ alcohol, such as citric acid esters, such as trioctyl citrate, triethyl citrate, acetyl tributyl citrate or tributyl citrate.
esters of a C₂-C₈ polyol and of one or more C₂-C₈ carboxylic acids, such as glycol diesters of monoacids, such as neopentyl glycol diheptanoate, or glycol triesters of monoacids, such as triacetin.

### Polar or Apolar Non-Volatile Hydrocarbon-Based Second Oils

The term “hydrocarbon-based oil” means an oil formed essentially from, or even constituted of, carbon and hydrogen atoms, and optionally of oxygen and nitrogen atoms, and not containing any silicon or fluorine atoms. These oils are thus different from silicone oils and fluoro oils.

The hydrocarbon-based oil may contain alcohol, ester, ether, carboxylic acid, amine and/or amide groups.

It should also be noted that the term “oil” denotes a non-aqueous, water-immiscible compound. The term “immiscible” means that the mixing of the same amount of water and oil, after stirring, does not result in a stable solution comprising only a single phase, under the abovementioned temperature and pressure conditions. Observation is performed by eye or using a phase-contrast microscope, if necessary, on 100 g of mixture obtained after sufficient stirring with a Rayneri blender to produce a vortex within the mixture (as a guide, 200 to 1000 rpm), the resulting mixture being left to stand, in a closed flask, for 24 hours at room temperature before observation.

The second non-volatile hydrocarbon-based oils may be chosen from polar non-volatile hydrocarbon-based oils different from the non-volatile hydrocarbon-based first oils described previously, or apolar non-volatile hydrocarbon-based oils.

### Second Polar Non-Volatile Hydrocarbon-Based Oils

Preferably, the second polar non-volatile hydrocarbon-based oil(s) comprise only carbon, hydrogen and oxygen atoms.

In particular, the second non-volatile hydrocarbon-based oil(s) are chosen from ester oils containing at least 17 carbon atoms, in particular containing between 17 and 70 carbon atoms; oils comprising at least one carbonate function; and mixtures thereof.

As regards the ester oils containing at least 18 carbon atoms, mention may be made of monoesters, diesters or triesters.

The ester oils may be hydroxylated or non-hydroxylated.

Thus, as second non-volatile hydrocarbon-based polar oil that is suitable for use, mention may be made of:
monoesters comprising at least 18 carbon atoms, in particular comprising between 18 and 40 carbon atoms in total, in particular the monoesters of formula R1COOR2 in which R1 represents a saturated or unsaturated, linear or branched or aromatic fatty acid residue comprising from 4 to 40 carbon atoms and R2 represents a hydrocarbon-based chain, which is in particular branched, containing from 4 to 40 carbon atoms, on condition that R1+R2≥18, for instance Purcellin oil (cetostearyl octanoate), isononyl isononanoate, C₁₂ to C₁₅ alcohol benzoate, 2-ethylhexyl palmitate, octyldodecyl neopentanoate, 2-octyldodecyl stearate, 2-octyldodecyl erucate, isostearyl isostearate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate or 2-octyldodecyl myristate.

Preferably, they are esters of formula R₁COOR₂ in which R₁ represents a linear or branched fatty acid residue containing from 4 to 40 carbon atoms and R₂ represents a hydrocarbon-based chain that is in particular branched, containing from 4 to 40 carbon atoms, R₁ and R₂ being such that R₁+R₂≥18.

Even more particularly, the ester comprises between 18 and 40 carbon atoms in total.

Preferred monoesters that may be mentioned include isononyl isononanoate, oleyl erucate and/or 2-octyldodecyl neopentanoate;
monoesters of a fatty acid comprising at least 18 carbon atoms, and in particular containing from 18 to 22 carbon atoms, and of diols. They may in particular be esters of lanolic acid, oleic acid, lauric acid or stearic acid, and of diols, for instance propylene glycol monoisostearate.
diesters comprising at least 17 carbon atoms, which diesters are optionally hydroxylated, linear or branched, saturated, unsaturated or aromatic diesters especially comprising between 17 and 60 carbon atoms in total, in particular between 17 and 50 carbon atoms in total. Use may thus be made more particularly of optionally hydroxylated diesters of dicarboxylic acid and of saturated or unsaturated monoalcohols, preferably such as diisostearyl malate. Use may also be made of diesters of glycol especially of C₂-05, of glycerol or of diglycerol and of linear or branched, saturated, unsaturated or aromatic monocarboxylic acids, such as neopentyl glycol dicaprate, propylene glycol dioctanoate, propylene glycol dibenzoate, diethylene glycol diisononanoate, or poly(2-glyceryl) diisostearate (especially such as the compound sold under the commercial reference Dermol DGDIS by the company Alzo).
hydroxylated monoesters and diesters comprising at least 18 carbon atoms, preferably with a total carbon number ranging from 18 to 70, for instance poly(3-glyceryl) diisostearate, isostearyl lactate, octyl hydroxystearate, octyldodecyl hydroxystearate, diisostearyl malate or glyceryl stearate.
triesters comprising at least 35 carbon atoms, especially comprising between 35 and 70 carbon atoms in total, in particular such as triesters of tricarboxylic acid, such as triisostearyl citrate, or tridecyl trimellitate, or glycol triesters of monocarboxylic acids such as polyglyceryl-2 triisostearate.
tetraesters comprising at least 35 carbon atoms, especially with a total carbon number ranging from 35 to 70, such as pentaerythritol or polyglycerol tetraesters of a monocarboxylic acid, for instance pentaerythrityl tetrapelargonate, pentaerythrityl tetraisostearate, pentaerythrityl tetraisononanoate, glyceryl tris(2-decyl)tetradecanoate, poly(2-glyceryl) tetraisostearate or pentaerythrityl tetrakis(2-decyl)tetradecanoate.
polyesters obtained by condensation of dimer and/or trimer of unsaturated fatty acid and of diol, such as those described in patent application FR 0 853 634, in particular such as dilinoleic acid and 1,4-butanediol. Mention may especially be made in this respect of the polymer sold by Biosynthis under the name Viscoplast 14436H (INCI name: dilinoleic acid/butanediol copolymer), or else copolymers of polyols and of dimer diacids, and esters thereof, such as Hailucent ISDA.
esters and polyesters of diol dimer and of monocarboxylic or dicarboxylic acid, such as esters of diol dimer and of fatty acid and esters of diol dimer and of dicarboxylic acid dimer, in particular which may be obtained from a dicarboxylic acid dimer derived in particular from the dimerization of an unsaturated fatty acid especially of C₈ to C₃₄, especially of C₁₂ to C₂₂, in particular of C₁₆ to C₂₀ and more particularly of C₁₈, such as esters of dilinoleic diacids and of dilinoleic diol dimers, for instance those sold by the company Nippon Fine Chemical under the trade names Lusplan DD-DA5® and DD-DA7®.
Polyesters resulting from the esterification of at least one hydroxylated carboxylic acid triglyceride by an aliphatic monocarboxylic acid and by an aliphatic dicarboxylic acid, which is optionally unsaturated, such as the succinic acid and isostearic acid castor oil sold under the reference Zenigloss by Zenitech.
hydrocarbon-based plant oils such as fatty acid triglycerides (which are liquid at room temperature), especially of fatty acids containing from 7 to 40 carbon atoms, such as heptanoic or octanoic acid triglycerides or jojoba oil; mention may be made in particular of saturated triglycerides such as caprylic/capric triglyceride and mixtures thereof, for instance the product sold under the reference Myritol 318 from Cognis, glyceryl triheptanoate, glyceryl trioctanoate, and C₁₈₋₃₆ acid triglycerides such as those sold under the reference Dub TGI 24 by Stéarineries Dubois, and unsaturated triglycerides such as castor oil, olive oil, ximenia oil or pracaxi oil.
sucrose esters, preferably chosen from hydrocarbon-based esters of sucrose and of a C₂-C₆ carboxylic acid, in particular those chosen from mixtures of esters of acetic acid and of isobutyric acid with sucrose, preferably sucrose diacetate hexakis(2-methylpropanoate), especially the compound whose INCI name is Sucrose acetate isobutyrate (sold especially under the reference Sustane SAIB Food Grade Kosher by the company Eastman Chemicals) and the compounds of INCI name Sucrose polysoyate sold under the reference Crodaderm S by the company Croda, sucrose polybehenate sold under the reference Crodaderm B by the company Croda, sucrose polycottonseedate sold under the reference Crodaderm C by the company Croda; and mixtures thereof
vinylpyrrolidone/1-hexadecene copolymers, for instance the product sold under the name Antaron V-216 (also known as Ganex V216) by the company ISP.
dialkyl carbonates, the two alkyl chains possibly being identical or different, such as the dicaprylyl carbonate sold under the name Cetiol CC® by Cognis
and mixtures thereof.

### Second Apolar Non-Volatile Hydrocarbon-Based Oils

The one or more second apolar non-volatile hydrocarbon-based oils are more particularly chosen from compounds comprising only carbon and hydrogen atoms.

Said linear or branched oils may be of mineral or synthetic origin, for instance:
liquid paraffin or derivatives thereof,
squalane,
isoeicosane,
naphthalene oil,
hydrogenated or non-hydrogenated polybutenes, for instance Indopol H-100, Indopol H-300 or Indopol H-1500 sold or manufactured by the company Amoco,
polyisobutenes and hydrogenated polyisobutenes, for instance the Parleam® products sold by the company Nippon Oil Fats, Panalane H-300 E sold by the company Amoco, Viseal 20000 sold by the company Synteal, Rewopal PIB 1000 sold by the company Witco or Parleam Lite sold by NOF Corporation,
decene/butene copolymers and polybutene/polyisobutene copolymers, especially Indopol L-14,
polydecenes and hydrogenated polydecenes, for instance Puresyn 10, Puresyn 150 or Puresyn 6 sold by ExxonMobil Chemical,
and mixtures thereof.

In accordance with a more particular embodiment of the invention, if the composition contains any, the content of second non-volatile hydrocarbon-based oil(s) is such that the non-volatile silicone oil(s)/second non-volatile hydrocarbon-based oil(s) weight ratio is greater than 1, preferably greater than 2.

If the composition comprises at least one second polar or apolar non-volatile hydrocarbon-based oil, the content of such oils is advantageously between 2 and 35% by weight, preferably from 8 to 30% by weight, relative to the weight of the composition.

### Volatile Oils

The composition according to the invention may optionally comprise at least one volatile oil, more particularly chosen from hydrocarbon-based or silicone volatile oils.

The term “volatile oil” means an oil with a non-zero vapour pressure, at room temperature and atmospheric pressure, ranging in particular from 0.13 Pa to 40 000 Pa, in particular ranging up to 13 000 Pa and more particularly ranging up to 1300 Pa.

The volatile hydrocarbon-based oils are preferably chosen from apolar hydrocarbon-based oils and may in particular be chosen from volatile hydrocarbon-based oils containing from 8 to 16 carbon atoms and mixtures thereof, and especially:
branched C₈-C₁₆alkanes such as C₈-C₁₆isoalkanes (also known as isoparaffins), isododecane, isodecane and isohexadecane, and, for example, the oils sold under the trade name Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture (Cetiol UT), the mixtures of n-undecane (C11) and of n-tridecane (C13) obtained in Examples 1 and 2 of patent application WO 2008/155 059 from the company Cognis, and
mixtures thereof.

The volatile silicone oil may be chosen from linear, branched or cyclic silicone oils, such as polydimethylsiloxanes (PDMSs) containing from 3 to 7 silicon atoms.

Examples of such oils that may be mentioned include octyl trimethicone, hexyl trimethicone, methyl trimethicone, decamethylcyclopentasiloxane, octamethylcyclotetrasiloxane, dodecamethylcyclohexasiloxane, decamethyltetrasiloxane, polydimethylsiloxanes such as those sold under the reference DC 200 (1.5 cSt), DC 200 (5 cSt) or DC 200 (3 cSt) by Dow Corning or KF 96 A from Shin-Etsu; alone or as mixtures.

If the composition comprises any, the content of volatile oil(s) is such that the silicone resin/volatile oil(s) weight ratio is greater than or equal to 1 and preferably greater than 1.

More particularly, if the composition comprises any, the content of volatile oil(s) is less than 30% by weight relative to the weight of the composition.

Preferably, the content of volatile oil(s) ranges from 0 to less than 30% by weight, more particularly from 0.5% to 20% by weight and preferably from 1% to 15% by weight, relative to the weight of the composition.

### Nonionic Surfactants

The composition according to the invention may comprise at least one nonionic hydrocarbon-based or silicone surfactant, and mixtures thereof.

The choice of surfactant(s) depends on the desired nature of the emulsion.

In particular, if an oil-in-water emulsion is envisaged, the surfactant(s) will preferably be at least chosen from nonionic hydrocarbon-based or silicone surfactants with an HLB (hydrophilic-lipophilic balance) value within the meaning of Griffin as defined in J. Soc. Cosm. Chem. 1954 (volume 5), pages 249-256, of greater than or equal to 8, and preferably at least from among hydrocarbon-based surfactants.

If a water-in-oil emulsion is moreover envisaged, then the surfactant(s) will preferably be chosen from nonionic hydrocarbon-based or silicone surfactants with an HLB value (as defined above) of less than 8, or mixtures thereof.

Nonionic Hydrocarbon-Based Surfactants with an HLB of Greater than or Equal to 8

According to a first particular embodiment of the invention, the composition comprises at least one nonionic hydrocarbon-based surfactant with an HLB value of greater than or equal to 8.

The nonionic surfactant(s) may be chosen especially from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof. Preferably, the non-ionic surfactant(s) may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and mixtures thereof.

1) Alkyl and polyalkyl esters of poly(ethylene oxide) that are preferably used are those containing at least one C₈-C₃₀ alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.

2) Alkyl and polyalkyl ethers of poly(ethylene oxide) that are preferably used are those containing at least one C₈-C₃₀ alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.

3) Optionally polyoxyethylenated alkyl and polyalkyl esters of sorbitan that are preferably used are those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

4) Optionally polyoxyethylenated alkyl and polyalkyl ethers of sorbitan that are preferably used are those with a number of ethylene oxide (EO) units ranging from 0 to 100.

5) Alkyl and polyalkyl glucosides or polyglucosides that are preferably used are those containing an alkyl group comprising from 6 to 30 carbon atoms and preferably from 6 to 18 or even from 8 to 16 carbon atoms, and containing a glucoside group preferably comprising from 1 to 5 and especially 1, 2 or 3 glucoside units. The alkylpolyglucosides may be chosen, for example, from decylglucoside (C₉/C₁₁-alkylpolyglucoside (1.4)), for instance the product sold under the name Mydol 10® by the company Kao Chemicals or the product sold under the name Plantacare 2000 UP® by the company Henkel and the product sold under the name Oramix NS 10® by the company SEPPIC; caprylyl/capryl glucoside, for instance the product sold under the name Plantacare KE 3711® by the company Cognis or Oramix CG 110® by the company SEPPIC; laurylglucoside, for instance the product sold under the name Plantacare 1200 UP® by the company Henkel or Plantaren 1200 N® by the company Henkel; cocoglucoside, for instance the product sold under the name Plantacare 818 UP® by the company Henkel; caprylylglucoside, for instance the product sold under the name Plantacare 810 UP® by the company Cognis; and mixtures thereof.

6) Examples of alkyl and polyalkyl esters of sucrose that may be mentioned are Crodesta F150, sucrose monolaurate sold under the name Crodesta SL 40, and the products sold by Ryoto Sugar Ester, for instance sucrose palmitate sold under the reference Ryoto Sugar Ester P1670, Ryoto Sugar Ester LWA 1695 or Ryoto Sugar Ester 01570. Sucrose monooleate, monomyristate and monostearate are also suitable for use.

7) Optionally (poly)oxyethylenated alkyl and polyalkyl esters of glycerol that are preferably used are those with a number of ethylene oxide (EO) units ranging from 0 to 100 and a number of glycerol units ranging from 1 to 30. Mention may be made, for example, of hexaglyceryl monolaurate, PEG-30 glyceryl stearate, polyglyceryl-2 laurate, polyglyceryl-10 laurate, polyglyceryl-10 stearate, polyglyceryl-10 oleate, PEG-7 glyceryl cocoate and PEG-20 glyceryl isostearate.

8) Optionally (poly)oxyethylenated alkyl and polyalkyl ethers of glycerol that are preferably used are those with a number of ethylene oxide (EO) units ranging from 0 to 100 and a number of glycerol units ranging from 1 to 30. Examples that may be mentioned include Nikkol Batyl Alcohol 100 and Nikkol Chimyl Alcohol 100.

Nonionic Hydrocarbon-Based Surfactants with an HLB of Less than 8

According to a second particular embodiment of the invention, the composition according to the invention may comprise at least one nonionic hydrocarbon-based surfactant chosen from surfactants with an HLB value of less than 8.

Examples of such surfactants that may especially be mentioned, alone or as mixtures, include:

1) (C₈-C₃₀)alkyl ethers of poly(ethylene oxide) preferably such as those with a number of ethylene oxide (EO) units ranging from 2 to 4. Examples that may especially be mentioned include laureth-2; steareth-2; oleth-2; ceteth-2; ceteareth-3;

2) (C₈-C₃₀)alkyl and (C₈-C₃₀)polyalkyl esters of ethylene oxide, of propylene oxide, of poly(ethylene oxide) or of poly(propylene oxide), preferably such as those with a number of ethylene oxide (EO) units ranging from 1 to 5, with for example glycol distearate, glycol stearate, PEG-2 oleate; PEG-3 oleate; PEG-4 dilaurate (HLB 6), propylene glycol isostearate; PEG-2.5 castor oil; PEG-3 castor oil;

3) as other surfactants that may be used, mention may be made of polyoxyalkylenated C₁₂-C₂₀ fatty acid polyesters, which are preferably polyhydroxylated, containing from 4 to 50 mol of ethylene oxide, having water-in-oil emulsifying properties. In particular, these polymers are block polymers, preferably of ABA structure, comprising poly(hydroxylated ester) blocks and polyethylene glycol blocks. The fatty acid of said emulsifying polymer as defined above preferably contains from 14 to 18 carbon atoms. The esters may be chosen especially from oleates, palmitates and stearates. The polyethylene glycol blocks of said emulsifying polymer as defined above preferably comprise from 20 to 40 mol of ethylene oxide. A polymeric surfactant that is particularly suitable for preparing the compositions of the invention is polyethylene glycol dipolyhydroxystearate containing 30 EO, sold under the trade name Arlacel P 135 by the company Croda.

4) (C₈-C₃₀)alkyl and (C₈-C₃₀)polyalkyl esters of sorbitan, for instance sorbitan trioleate, sorbitan sesquioleate, sorbitan oleate, sorbitan palmitate, sorbitan stearate, sorbitan isostearate, mixtures of sorbitan stearate and sucrose cocoate (Arlacel 2121 sold by the company Croda), or sorbitan glyceryl isostearate; sorbitan isostearate mixed with hydrogenated castor oil, stearic acid and white wax (Arlacel 986 sold by the company Croda), and mixtures thereof.

5) (C₈-C₃₀)alkyl and poly(C₈-C₃₀)alkyl esters of (poly)glycerol that are preferably used are those with a number of glycerol units ranging from 1 to 4. Mention may be made, for example, of polyglyceryl-4 isostearate (Isolan GI 34 sold by the company Evonik Goldschmidt); polyglyceryl-2 sesquiisostearate, polyglyceryl-3 diisostearate (Lameform TGI sold by the company Cognis), glyceryl isostearate, glyceryl stearate, glyceryl laurate, alone or as mixtures.

### Silicone Surfactants

In accordance with a third embodiment of the invention, the composition comprises at least one silicone surfactant, which is in particular nonionic. The silicone surfactant(s) may be chosen from compounds with an HLB value of greater than or equal to 8, or less than 8.

Among the nonionic silicone surfactants that are suitable for use, mention may be made of alkyl or alkoxy dimethicone copolyols bearing an alkyl or alkoxy chain on the side or at the end of the silicone backbone, containing, for example, from 6 to 22 carbon atoms; dimethicone copolyols, which are more particularly oxypropylenated and/or oxyethylenated polydimethyl methyl siloxanes, branched (poly)glycerolated polysiloxanes, and also crosslinked elastomeric solid organopolysiloxanes comprising at least one oxyalkylene group, and mixtures thereof.

Examples of alkyl or alkoxy dimethicone copolyols that may be mentioned include the compounds of formula (I) below:

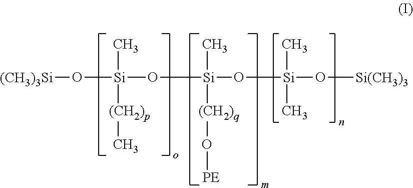

in which:
PE represents (—C₂H₄O)ₓ—(C₃H₆O)_{y}—R, R being chosen from a hydrogen atom and an alkyl radical of 1 to 4 carbon atoms, x ranging from 0 to 100 and y ranging from 0 to 80, x and y not simultaneously being 0; preferably, R represents a hydrogen atom;
m ranging from 1 to 40, preferably from 1 to 10;
n ranging from 10 to 200, preferably from 10 to 100;
o ranging from 1 to 100, preferably from 1 to 30;
p ranging from 5 to 21, more particularly from 7 to 21;
q ranging from 0 to 4, preferably from 1 to 3.

As examples of dimethicone copolyols, use may be made of those corresponding more particularly to formula (II) below:

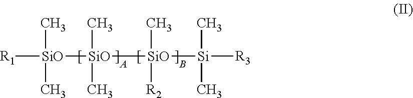

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, a C₁-C₃ alkyl radical or a C₂-C₄ acyl radical;
A is an integer ranging from 0 to 200;
B is an integer ranging from 0 to 50; on condition that A and B are not simultaneously equal to zero;
x is an integer ranging from 1 to 6;
y is an integer ranging from 1 to 30; and
z is an integer ranging from 0 to 30, preferably ranging from 0 to 20.

Among the silicone surfactants that are particularly preferred, mention may be made of:
dimethicone copolyols, for instance those sold under the names KF-6015 (PEG-3 dimethicone), KF-6016 (PEG-9 methyl ether dimethicone), KF-6017 (PEG-10 dimethicone), KF-6028 (PEG-9 polydimethylsiloxyethyl dimethicone), KF-6050 L (PEG/PPG 18/18 dimethicone in cyclopentasiloxane), X-22-6711D (PEG/PPG 18/18 dimethicone in dimethicone), sold by the company Shin-Etsu; the dimethicone copolyols sold under the names Xiameter OFX-0193 Fluid (PEG-12 dimethicone); Dow Corning 3225C® (PEG/PPG-18/18 dimethicone in a mixture of cyclotetrasiloxane and cyclopentasiloxane), DC 5225 C Formulation Aid (PEG/PPG-18/18 dimethicone in cyclopentasiloxane); or the product sold under the name SF 1528 GE (mixture of PEG/PPG-20/15 dimethicone and of cyclopentasiloxane) by Momentive Performance Materials.

Use may be made of the product Abil Care 85 (bis-PEG/PPG-16/16 PEG/PPG-16/16 dimethicone as a mixture with capric/caprylic acid triglyceride).

Use may also be made of alkyl dimethicone copolyols such as lauryl PEG/PPG-18/18 methicone (which is more particularly an alkoxylated derivative of lauryl methicone containing on average 18 mol of ethylene oxide and 18 mol of propylene oxide, sold under the name Dow Corning 5200 Formulation Aid by the company Dow Corning; cetyl PEG/PPG-10/1 dimethicone (which is more particularly a copolymer of cetyl dimethicone and of an alkoxylated derivative of dimethicone containing on average 10 mol of ethylene oxide and 1 mol of propylene oxide) such as the product sold under the name Abil EM 90 by the company Evonik Goldschmidt and also the mixture of cetyl PEG/PPG-10/1 dimethicone, of polyglyceryl isostearate (4 mol) and of hexyl laurate sold under the name Abil WE 09 by the company Evonik Goldschmidt.

Use may also be made of polyglyceryl-3 disiloxane dimethicone (KF 6100 from Shin-Etsu).

Emulsion surfactants that may also be mentioned include, in particular for water-in-oil emulsions, crosslinked elastomeric solid organopolysiloxanes comprising at least one oxyalkylene group, such as the products obtained according to the procedure of Examples 3, 4 and 8 of U.S. Pat. No. 5,412,004 and the examples of U.S. Pat. No. 5,811,487, especially the product of Example 3 (synthetic example) of U.S. Pat. No. 5,412,004, and such as the product sold under the references KSG 21 and KSG-210 by the company Shin-Etsu.

Preferably, the C₈-C₂₂ alkyl dimethicone copolyol that is used is cetyl dimethicone copolyol, especially the product whose INCI name is Cetyl PEG/PPG-10/1 Dimethicone, for instance the product sold under the name Abil EM-90 by the company Evonik Goldschmidt. Use may also be made of a mixture of cetyl dimethicone copolyol with polyglyceryl-4 isostearate and hexyl laurate, for instance the product sold under the name Abil WE-09 by the company Evonik Goldschmidt (the INCI name is polyglyceryl-4 isostearate (and) hexyl laurate (and) cetyl PEG/PPG-10/1 dimethicone).

Advantageously, if they are present, the silicone surfactant(s) are chosen from dimethicone copolyols, the alkyl dimethicone copolyols described previously, alone or as mixtures.

More particularly, the silicone surfactant(s) are chosen from C8-C22 alkyl dimethicone copolyols such as cetyl dimethicone copolyol (INCI name: Cetyl PEG/PPG-10/1 Dimethicone), dimethicone copolyols such as, for example, PEG dimethicone, PEG/PPG 18/18 dimethicones (INCI name), and also mixtures thereof. Use may also be made of a mixture of cetyl dimethicone copolyol with polyglyceryl-4 isostearate and hexyl laurate, for instance the product sold under the name Abil WE-09 by the company Evonik Goldschmidt (the INCI name is polyglyceryl-4 isostearate (and) hexyl laurate (and) cetyl PEG/PPG-10/1 dimethicone).

Advantageously, in the case where the composition comprises at least one nonionic surfactant, the content of this type of compound ranges from 0.1% to 15% by weight and preferably from 0.5% to 10% by weight, relative to the total weight of the composition.

Preferably, the composition according to the invention comprises at least one nonionic hydrocarbon-based surfactant.

According to an even more preferred embodiment, the composition according to the invention comprises at least one nonionic hydrocarbon-based surfactant with an HLB value of greater than or equal to 8.

In accordance with this preferred embodiment of the invention, the composition may also comprise, as additional surfactant(s), at least one nonionic hydrocarbon-based surfactant whose HLB value is less than 8; at least one nonionic silicone surfactant, preferably with an HLB value of less than 8; and also mixtures thereof.

In the case where such additional surfactants are present, their content is preferably such that the composition is in the form of an oil-in-water emulsion.

### Additional Ionic Surfactants

The composition may optionally comprise one or more ionic, in particular anionic, surfactants, which are preferably hydrocarbon-based.

These surfactants may be chosen from salts of alkali metals especially such as sodium or potassium, or alternatively of primary or secondary amine or alkanolamine, which is in particular of C₂-C₄, of the following compounds:

These compounds generally comprise from 8 to 30 carbon atoms, in particular from 8 to 20 carbon atoms, in their longest hydrocarbon-based chain, and are saturated or unsaturated, and linear, branched or cyclic. They may also comprise up to 20 oxyalkylene units, preferably up to 15 units (in particular oxyethylene units):

alkyl ether sulfates,

salts of fatty acids especially of C₈-C₂₀, in particular monocarboxylic salts;

carboxylates such as N-acylamino acids, alkyl glycol carboxylates, ether carboxylates, amido ether carboxylates;

amino acid salts, in particular sarcosinates, alaninates, glutamates, aspartates and glycinates,

sulfonates, such as alpha-olefin sulfonates, in particular alkanolamine or alkali metal (especially such as sodium) salts of dodecylbenzene sulfonate,

isethionates, such as acyl isethionates,

taurates, such as N-acyl methyl taurates, in particular N-acyl methyl taurates,

sulfosuccinates, such as alkyl sulfosuccinates, especially dioctyl sulfosuccinate salts,

alkylsulfoacetates,

phosphates and alkyl phosphates,

polypeptides obtained, for example, by condensation of a fatty chain onto amino acids from cereals and especially from wheat and oat.

Preferably, when it comprises any, the composition comprises not more than 2.5% by weight, more particularly not more than 1.5% by weight, preferably not more than 1% by weight, or even not more than 0.5% by weight, relative to the weight of the composition, of salts of alkali metals, of amine or alkanolamine, or of fatty acids, especially of C₈-C₂₀.

Advantageously, when they are present, the composition comprises not more than 2.5% by weight, more particularly not more than 1.5% by weight, preferably not more than 1% by weight, or even not more than 0.5% by weight, relative to the weight of the composition, of anionic hydrocarbon-based surfactant(s).

### Aqueous Phase

The composition according to the invention comprises at least 10% by weight of water, relative to the weight of the composition.

Advantageously, the water content is between 10% and 70% by weight, preferably between 15% and 65% by weight, in particular between 20% and 60% by weight, relative to the weight of the composition.

The composition in accordance with the invention may comprise, besides water, at least one water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the compositions according to the invention may also be volatile.

Among the water-soluble solvents that may be used in the compositions in accordance with the invention, mention may be made especially of lower monoalcohols containing from 1 to 5 carbon atoms, such as ethanol and isopropanol, C₃ and C₄ ketones and C₂-C₄ aldehydes.

Preferably, the composition according to the invention preferably comprises a total content of monoalcohols comprising between 2 and 8 carbon atoms of between 0 and 15% by weight (limits inclusive) relative to the total weight of the composition.

Preferably, the composition according to the invention comprises a total content of monoalcohols comprising between 2 and 8 carbon atoms of between 0 and 10% by weight (limits inclusive), advantageously between 0 and 5% by weight (limits inclusive) relative to the total weight of the composition.

Preferably, the composition according to the invention is free of monoalcohols comprising between 2 and 8 carbon atoms.

Preferably, said monoalcohol(s) comprising between 2 and 8 carbon atoms are chosen from ethanol, butanol, methanol and isopropanol.

C₂-C₈ Polyol

The composition may also comprise at least one saturated or unsaturated, linear or branched C₂-C₈ and preferably C₃-C₆ polyol, comprising from 2 to 6 hydroxyl groups.

Preferably, the polyol is chosen from glycerol, propylene glycol, 1,3-butylene glycol, dipropylene glycol, dibutylene glycol and diglycerol, and mixtures thereof.

Preferably, the polyol content represents less than 10% by weight, more particularly from 0.05% to less than 10% by weight, preferably from 0.1% to less than 10% by weight and more particularly from 1% to 6% by weight, relative to the weight of the composition.

### Hydrophilic Thickeners

The composition according to the invention may comprise at least one hydrophilic thickening polymer (also known as an aqueous-phase-thickening polymer).

More particularly, this thickening polymer may be chosen from:
homopolymers or copolymers of acrylic or methacrylic acid or salts thereof and esters thereof. Examples that may be mentioned in particular include the products sold under the names Versicol F or Versicol K by the company Allied Colloid, Ultrahold 8 by the company Ciba-Geigy, Cosmedia SP by the company BASF, Lecigel (mixture of sodium acrylates copolymer/lecithin) by the company Lucas Meyer Cosmetics; polyacrylic acids of Synthalen K type, and salts, especially sodium salts, of polyacrylic acid (corresponding to the INCI name sodium acrylate copolymer) and more particularly a crosslinked sodium polyacrylate (corresponding to the INCI name sodium acrylate copolymer (and) caprylic/capric triglyceride) sold under the name Luvigel EM.

Mention may also be made of polyacrylic acid/alkyl acrylate copolymers, preferably modified or unmodified carboxyvinyl polymers, most particularly acrylate/C10-C30-alkyl acrylate copolymers (INCI name: Acrylate/C10-30 Alkyl Acrylate Crosspolymer) such as the products sold by the company Lubrizol under the trade names Pemulen TR1, Pemulen TR2, Carbopol 1382, Carbopol EDT 2020 and even more preferentially Pemulen TR-2; copolymers of methacrylic acid, of methyl methacrylate, of methylstyrene isocyanate and of PEG-40 behenate (INCI name: polyacrylate-3) (Viscophobe DB 1000 sold by the company Dow),
copolymers of acrylic acid and of acrylamide sold in the form of the sodium salt thereof under the Reten names by the company Hercules, the sodium polymethacrylate sold under the name Darvan No. 7 by the company Vanderbilt, and the sodium salts of polyhydroxycarboxylic acids sold under the name Hydagen F by the company Henkel,
homopolymers and copolymers based on acrylamidopropanesulfonic acid, for instance:
   polyacrylamidomethylpropanesulfonic acid partially neutralized with aqueous ammonia and highly crosslinked, for example the product Hostacerin AMPS sold in particular by the company Clariant,
   copolymers of acrylamidopropanesulfonic acid/acrylamide for example of Sepigel or Simulgel type sold especially by the company SEPPIC,
   polyoxyethylenated acrylamidopropanesulfonic acid/alkyl methacrylate copolymers (crosslinked or non-crosslinked) of the type such as, inter alia, Aristoflex HMS and Aristoflex TAC, sold by the company Clariant,
   copolymers of acrylamidomethylpropanesulfonic acid and of hydroxyethyl acrylate, for instance the acrylamidomethylpropanesulfonic acid/hydroxyethyl acrylate copolymer especially such as the product used in the commercial product sold under the name Simulgel NS by the company SEPPIC, or the acrylamido methylpropanesulfonic acid/hydroxyethyl acrylate copolymer especially such as the product used in the commercial product sold under the name Sepinov EMT 10 sold by the company SEPPIC (INCI name: Hydroxyethyl acrylate/sodium acryloyldimethyl taurate copolymer);
   copolymers of acrylamidomethylpropanesulfonic acid and of vinylpyrrolidone, such as the product Aristoflex AVC (ammonium acryloyldimethyl taurate/VP copolymer in water) sold by the company Clariant;
and mixtures thereof.

Other examples of hydrophilic gelling polymers that may be mentioned include:
anionic, cationic, amphoteric or nonionic chitin or chitosan polymers;
cellulose polymers, in particular (C₁-C₃)hydroxyalkylcelluloses, chosen from hydroxyethylcellulose, hydroxypropylcellulose, hydroxypropylmethylcellulose, hydroxymethylcellulose, ethylhydroxyethylcellulose; carboxymethylcellulose;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol,
optionally modified polymers of natural origin, such as galactomannans and derivatives thereof, for instance konjac gum, gellan gum, locust bean gum, fenugreek gum, karaya gum, gum tragacanth, gum arabic, acacia gum, guar gum, hydroxypropyl guar, hydroxypropyl guar modified with sodium methylcarboxylate groups (Jaguar XC97-1, Rhodia), xanthan gum and derivatives thereof;
alginates and carrageenans;
muccopolysaccharides such as hyaluronic acid;
and mixtures thereof.

According to a preferred embodiment, the thickening polymer is chosen from copolymers of acrylamidomethylpropanesulfonic acid and in particular from copolymers of acrylamidomethylpropanesulfonic acid and of hydroxyethyl acrylate, or mixtures thereof.

Preferably, when the composition contains any, the content of hydrophilic thickening polymer is between 0.01 and 1.5% by weight, preferably between 0.05 and 1% by weight and advantageously 0.1 to 0.8%, relative to the weight of the composition.

### Dyestuffs

A composition according to the invention preferably comprises at least one dyestuff Preferably, it is chosen from water-soluble or water-insoluble, liposoluble or non-liposoluble, organic or mineral dyestuffs, and materials with an optical effect, and mixtures thereof.

For the purposes of the present invention, the term “dyestuff” means a compound that is capable of producing a coloured optical effect when it is formulated in sufficient amount in a suitable cosmetic medium.

### Water-Soluble Dyestuffs

The water-soluble dyestuffs used according to the invention are more particularly water-soluble dyes.

For the purposes of the invention, the term “water-soluble dye” means any natural or synthetic, generally organic compound, which is soluble in an aqueous phase or water-miscible solvents and which is capable of imparting colour. In particular, the term “water-soluble” is intended to characterize the capacity of a compound to be dissolved in water, measured at 25° C., to a concentration at least equal to 0.1 g/l (production of a macroscopically isotropic, transparent, coloured or colourless solution). This solubility is in particular greater than or equal to 1 g/l.

As water-soluble dyes that are suitable for use in the invention, mention may be made in particular of synthetic or natural water-soluble dyes, for instance DC Red 6 (Lithol Rubine Na; CI: 15850), DC Red 22 (CI: 45380), DC Red 28 (CI: 45410 Na salt), DC Red 30 (CI: 73360), DC Red 33 (CI: 17200), FDC Red 40 (CI 16035), DC Orange 4 (CI: 15510 Na salt), FDC Yellow 5 (CI: 19140), FDC Yellow 6 (CI: 15985), DC Yellow 8 (CI: 45350 Na salt), FDC Green 3 (CI: 42053), DC Green 5 (CI: 61570), FDC Blue 1 (CI: 42090).

As non-limiting illustrations of sources of water-soluble dyestuff(s) that may be used in the context of the present invention, mention may be made in particular of those of natural origin, such as extracts of cochineal carmine, of beetroot, of grape, of carrot, of tomato, of annatto, of paprika, of henna, of caramel and of curcumin.

Thus, the water-soluble dyestuffs that are suitable for use in the invention are especially carminic acid, betanin, anthocyans, enocyanins, lycopene, bixin, norbixin, capxanthin, capsorubin, flavoxanthin, lutein, cryptoxanthin, rubixanthin, violaxanthin, riboflavin, rhodoxanthin, cantaxanthin and chlorophyll, and mixtures thereof.

They may also be copper sulfate, iron sulfate, water-soluble sulfopolyesters, rhodamine, betaine, methylene blue, the disodium salt of tartrazine and the disodium salt of fuchsin.

Some of these water-soluble dyestuffs are especially permitted for food use. Representatives of these dyes that may be mentioned more particularly include dyes of the carotenoid family, referenced under the food codes E120, E162, E163, E160a-g, E150a, E101, E100, E140 and E141.

### Liposoluble Dyestuffs

Among the liposoluble dyes, mention may be made particularly of dyes of fluoran type, for instance Sudan Red, FDC Red 4, DC Red 17, Red 21, Red 27, DC Green 6, β-carotene, Sudan Brown, Yellow 10, DC Yellow 11, DC Violet 2, DC Orange 4, DC Orange 5 and quinoline yellow, or mixtures thereof

### Pigments

The term “pigments” should be understood as meaning white or coloured, inorganic (mineral) or organic particles, which are insoluble in the medium, and which are intended to colour and/or opacify the composition and/or deposit produced with the composition.

The pigments may be chosen from mineral pigments, organic pigments and composite pigments (i.e. pigments based on mineral and/or organic materials).

The pigments may be chosen from monochromic pigments, lakes, nacres, and pigments with an optical effect, for instance reflective pigments and goniochromatic pigments.

The mineral pigments may be chosen from metal oxide pigments, chromium oxides, iron oxides, titanium dioxide, zinc oxides, cerium oxides, zirconium oxides, manganese violet, Prussian blue, ultramarine blue and ferric blue, and mixtures thereof.

The organic pigments may be, for example:
cochineal carmine,
organic pigments of azo dyes, anthraquinone dyes, indigoid dyes, xanthene dyes, pyrene dyes, quinoline dyes, triphenylmethane dyes or fluoran dyes,
organic lakes or insoluble sodium, potassium, calcium, barium, aluminium, zirconium, strontium or titanium salts of acidic dyes such as azo, anthraquinone, indigoid, xanthene, pyrene, quinoline, triphenylmethane or fluoran dyes. These dyes generally comprise at least one carboxylic or sulfonic acid group,
melanin-based pigments.

Among the organic pigments, mention may be made of D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 and FD&C Yellow No. 6.

The pigments may have undergone a hydrophobic treatment. The hydrophobic treatment agent may be chosen, for example, from silicones such as methicones, dimethicones and perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, amino acids; N-acylamino acids or salts thereof; lecithin, isopropyl triisostearyl titanate, and mixtures thereof.

The N-acylamino acids may comprise an acyl group containing from 8 to 22 carbon atoms, for instance a 2-ethylhexanoyl, caproyl, lauroyl, myristoyl, palmitoyl, stearoyl or cocoyl group. The salts of these compounds may be the aluminium, magnesium, calcium, zirconium, zinc, sodium or potassium salts. The amino acid may be, for example, lysine, glutamic acid or alanine.

The term “alkyl” mentioned in the compounds cited previously especially denotes an alkyl group containing from 1 to 30 carbon atoms and preferably containing from 5 to 16 carbon atoms.

Hydrophobic-treated pigments are described especially in patent application EP-A-1 086 683.

The pigments may also have undergone a hydrophilic treatment, for example with polyalkoxylated silicones.

It may also be envisaged to use pigments that are in a form dispersed in water, sorbitol, glycerol or glycols, alone or as mixtures, for instance the products of the Covarine, Covasorb and Covasop ranges from Sensient, the pigments of the WD series from Daito, and Worlée Base AQ from Worlée.

### Nacres

For the purposes of the present patent application, the term “nacre” means coloured particles of any form, which may or may not be iridescent, in particular produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, titanium mica coated with an organic dye in particular of the abovementioned type, and also nacreous pigments based on bismuth oxychloride. They may also be mica particles, at the surface of which are superposed at least two successive layers of metal oxides and/or of organic dyestuffs.

The nacres may more particularly have a yellow, pink, red, bronze, orangey, brown, gold and/or coppery colour and/or glint.

As illustrations of nacres that may be introduced as interference pigments into the first composition, mention may be made of the gold-coloured nacres sold in particular by the company Engelhard under the names Brilliant gold 212G (Timica), Gold 222C (Cloisonne), Sparkle gold (Timica), Gold 4504 (Chromalite) and Monarch gold 233X (Cloisonne); the bronze nacres sold in particular by the company Merck under the names Bronze fine (17384) (Colorona) and Bronze (17353) (Colorona) and by the company Engelhard under the name Super bronze (Cloisonne); the orange nacres sold in particular by the company Engelhard under the names Orange 363C (Cloisonne) and Orange MCR 101 (Cosmica) and by the company Merck under the names Passion orange (Colorona) and Matte orange (17449) (Microna); the brown nacres sold in particular by the company Engelhard under the names Nu-antique copper 340XB (Cloisonne) and Brown CL4509 (Chromalite); the nacres with a copper glint sold in particular by the company Engelhard under the name Copper 340A (Timica); the nacres with a red glint sold in particular by the company Merck under the name Sienna fine (17386) (Colorona); the nacres with a yellow glint sold in particular by the company Engelhard under the name Yellow (4502) (Chromalite); the red nacres with a gold glint sold in particular by the company Engelhard under the name Sunstone G012 (Gemtone); the pink nacres sold in particular by the company Engelhard under the name Tan opal G005 (Gemtone); the black nacres with a gold glint sold in particular by the company Engelhard under the name Nu antique bronze 240 AB (Timica), the blue nacres sold in particular by the company Merck under the name Matte blue (17433) (Microna), the white nacres with a silvery glint sold in particular by the company Merck under the name Xirona Silver, and the golden-green pinkish-orangey nacres sold in particular by the company Merck under the name Indian summer (Xirona), and mixtures thereof.

The content of dyestuffs is advantageously between 0.05% and 10% by weight and preferably between 0.05% and 5% by weight relative to the weight of the composition.

### Fillers

The composition according to the invention may comprise at least one filler, of organic or mineral nature.

The term “filler” should be understood as meaning colourless or white solid particles of any shape, which are in an insoluble form dispersed in the medium of the composition. These particles, of mineral or organic nature, give body or rigidity to the composition and/or softness and uniformity to the makeup. They are different from dyestuffs.

Among the fillers that may be used in the compositions according to the invention, mention may be made of silica, kaolin, starch, lauroyllysine, fumed silica particles, which are optionally hydrophilic-treated, mica, talc, sericite, polyamide (Nylon®) powder, poly-β-alanine powder and polyethylene powder, powders of tetrafluoroethylene polymers (Teflon®), hollow polymer microspheres such as those of polyvinylidene chloride/acrylonitrile, for instance Expancel® (Nobel Industries), acrylic acid copolymer microspheres, silicone resin microbeads (for example Tospearls® from Toshiba), polyorganosiloxane elastomer particles, precipitated calcium carbonate, magnesium carbonate, magnesium hydrogen carbonate, hydroxyapatite, barium sulfate, aluminium oxides, polyurethane powders, composite fillers, hollow silica microspheres, glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate and magnesium myristate, and mixtures thereof.

A composition used according to the invention may comprise one or more fillers in a content ranging from 0.1% to 10% by weight and in particular from 0.2% to 8% by weight relative to the total weight of the composition.

### Lipophilic Thickener

The hydrophobic thickener may be chosen from mineral thickeners especially such as organophilic clays; hydrophobic fumed silicas; hydrophobic silica aerogels; from organic thickeners such as oil-gelling polymers of the block polymer type, especially triblock or star polymers, resulting from the copolymerization of at least one styrene monomer and of at least one hydrocarbon monomer bearing one or two C₂-C₅ ethylenic unsaturations, such as ethylene, propylene, butadiene, isoprene and/or pentadiene, such as the polymers sold under the name Kraton; polyamide resins comprising alkyl groups containing from 12 to 22 carbon atoms, such as those described in U.S. Pat. No. 5,783,657; polysaccharide alkyl ethers, especially in which the alkyl group is of C₁-C₂₄, preferably C₁-C₁₀ and better still C₁-C₆, described especially in EP 898 958, and in particular alkyl guar gums (with a C₁-C₆ alkyl group), such as those described in EP 708 114; esters of dextrin and of a fatty acid, preferably of C₁₂ to C₂₄, in particular C₁₄-C₁₈, for example dextrin palmitate and dextrin myristate; N-acylglutamides in which the acyl group is a linear or branched C₈ to C₂₂ alkyl chain; preferably dialkyl N-acylglutamides, for instance lauroylglutamic acid dibutylamide or N-2-ethylhexanoyl glutamic acid dibutylamide; hydroxystearic acid; ethylenediamine stearyl dimer dilinoleate copolymer (sold, for example, under the name Oleocraft by Croda); and also mixtures thereof.

Preferably, when the composition comprises at least one hydrophobic thickener, this thickener is preferably chosen from mineral thickeners.

Clays are silicates containing a cation that may be chosen from calcium, magnesium, aluminium, sodium, potassium and lithium cations, and mixtures thereof. Examples of such products that may be mentioned include clays of the smectite family such as montmorillonites, hectorites, bentonites, beidellites and saponites, and also of the family of vermiculites, stevensite and chlorites. These clays may be of natural or synthetic origin. Organophilic clays are clays modified with a chemical compound chosen from quaternary amines, tertiary amines, amine acetates, imidazolines, amine soaps, fatty sulfates, alkyl aryl sulfonates and amine oxides, and mixtures thereof.

Mention may thus be made of hectorites modified with a quaternary amine, more specifically with a C₁₀ to C₂₂ fatty acid ammonium halide, such as a chloride, such as hectorite modified with distearyldimethylammonium chloride (CTFA name: Disteardimonium hectorite), for instance the product sold under the name Bentone 38V®, Bentone 38V CG or Bentone EW CE by the company Elementis, or stearalkonium hectorites, such as Bentone 27 V.

Mention may also be made of quaternium-18 bentonites, such as those sold under the names Bentone 34 by the company Elementis, Tixogel VP by the company United Catalyst and Claytone 40 by the company Southern Clay; stearalkonium bentonites, such as those sold under the names Tixogel LG by the company United Catalyst and Claytone AF and Claytone APA by the company Southern Clay; or quaternium-18/benzalkonium bentonites, such as that sold under the name Claytone HT by the company Southern Clay.

According to a preferred embodiment, the thickener is chosen from organophilic modified clays, in particular organophilic modified hectorites, in particular modified with stearylbenzyldimethylammonium halides, preferably chlorides, or with distearyldimethylammonium chloride.

The hydrophobic fumed silicas may be obtained by modification of the surface of the silica via a chemical reaction that generates a reduction in the number of silanol groups, these groups possibly being substituted especially with hydrophobic groups. The hydrophobic groups may be:
trimethylsiloxyl groups, which are obtained especially by treating fumed silica in the presence of hexamethyldisilazane. Silicas thus treated are known as Silica silylate according to the CTFA (6th Edition, 1995). They are sold, for example, under the references Aerosil R812® by the company Degussa, and Cab-O-Sil TS-530® by the company Cabot;
dimethylsilyloxyl or polydimethylsiloxane groups, which are especially obtained by treating fumed silica in the presence of polydimethylsiloxane or dimethyldichlorosilane. Silicas thus treated are known as Silica dimethyl silylate according to the CTFA (6th Edition, 1995). They are sold, for example, under the references Aerosil R972® and Aerosil R974® by the company Degussa, and Cab-O-Sil TS-610® and Cab-O-Sil TS-720® by the company Cabot.

Silica aerogels are porous materials obtained by replacing (by drying) the liquid component of a silica gel with air. They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York: Academic Press, 1990.

Use will preferably be made of hydrophobic silica aerogel particles surface-modified with trimethylsilyl groups.

Hydrophobic silica aerogels that may be mentioned, for example, include the aerogel sold under the name VM-2260 (INCI name: Silica silylate), by the company Dow Corning, the particles of which have a mean size of about 1000 microns and a specific surface area per unit mass ranging from 600 to 800 m²/g. Mention may also be made of the aerogels sold by the company Cabot under the references Aerogel TLD 201, Aerogel OGD 201, Aerogel TLD 203, Enova® Aerogel MT 1100 and Enova Aerogel MT 1200.

The elastomeric polyorganosiloxanes are generally partially or totally crosslinked and possibly of three-dimensional structure. The elastomeric polyorganosiloxanes combined with a fatty phase are generally in the form of a gel constituted of an elastomeric organopolysiloxane combined with a fatty phase, included in at least one hydrocarbon-based oil and/or one silicone oil. They may be chosen especially from the crosslinked polymers described in patent application EP-A-0 295 886. According to said patent application, the elastomeric organopolysiloxanes are obtained by addition reaction and crosslinking of at least:
(a) one organopolysiloxane bearing at least two lower alkenyl groups per molecule;
(b) one organopolysiloxane bearing at least two hydrogen atoms linked to a silicon atom per molecule; and
(c) a platinum-type catalyst.

The hydrophobic thickener may be present in a content ranging from 0.05% to 10% by weight and preferably ranging from 0.1% to 8% by weight relative to the total weight of the composition.

### Common Additives

The composition according to the invention may also comprise any common cosmetic ingredient that may be chosen especially from waxes, pasty compounds, additional moisturizers (also known as humectants) other than the abovementioned polyols, antioxidants, fragrances, menthol or menthol derivatives, preserving agents, neutralizers, sunscreens, sweeteners, vitamins, free-radical scavengers, sequestrants, salts (magnesium sulfate, sodium chloride), pH regulators, and mixtures thereof.

Additionally, the composition according to the invention is preferably free of alkylcellulose the alkyl residue of which comprises between 1 and 6 carbon atoms and preferably between 2 and 3 carbon atoms, preferably ethylcellulose

Needless to say, a person skilled in the art will take care to select the optional additional ingredients and/or the amount thereof such that the advantageous properties of the composition according to the invention are not, or are not substantially, adversely affected by the envisaged addition.

Another subject of the invention is a process for making up and/or caring for the lips, which consists in applying the composition according to the invention.

The composition according to the invention may be packaged in any type of device that is common in the field of fluid cosmetic compositions intended especially to be applied to the lips or the face (for instance the cheeks).

It may thus be envisaged to use devices containing a container comprising an applicator equipped with a ball (roll-on), a container of dispensing pen type, terminated with an end provided with at least one orifice through which the composition may be expelled, or alternatively terminated with a felt or with a flocked tip, or with a brush; a container comprising a dip applicator, for instance a brush.

Such devices may or may not be provided with a mechanism for dispensing the composition making it possible to expel said composition from the container to the application member, or to the support. It should be noted that this mechanism may advantageously comprise a means for metering out the composition.

The examples which follow serve to illustrate the invention without, however, limiting the scope thereof.

### EXAMPLES

### Example 1

The composition whose ingredients are listed in the table below is prepared (the percentages are expressed as weight of starting material, unless otherwise indicated).

| Ingredients (INCI name) | Content |
|---|---|
| Polysorbate 60 (Tween 60-LQ-(MV) from Croda) | 4% |
| Sodium dehydroacetate (Tristat SDHA FCC/NF from TRI-K) | 0.5% |
| Red 33 | 0.30% |
| Ammonium polyacryloyldimethyl taurate (Hostacerin AMPS | 0.45% |
| from Clariant) | |
| Silicone resin (trimethyl siloxysilicate (and) isododecane: | 44.5% |
| X-21-5616 from Shin-Etsu; in a silicone resin/isododecane | |
| weight ratio of 60/40) | |
| Trimethylsiloxyphenyl Dimethicone (Belsil PDM 1000 from | 20% |
| Wacker) | |
| Water | qs 100% |

### Procedure

The polysorbate 60 and water are heated at 50° C. with a deflocculator until the mixture is homogeneous, and the heating is stopped.

The dye is then added and the mixture is stirred until dissolution is complete.

The thickening polymer is then added with stirring until the mixture thickens without gel lumps.

The silicone resin and the isododecane are then poured slowly onto the mixture obtained previously while increasing the stirring speed, and stirring is continued for 10 minutes.

Finally, the trimethylsiloxyphenyl dimethicone is added slowly and stirring is maintained for 10 minutes.

### Evaluation of the Composition

A stable, homogeneous direct emulsion is obtained. The composition is centrifuged for 1 hour at 900×g according to the protocol detailed in the description. Slight phase separation as a 4 mm pellet (bottom of the container) is obtained.

The composition is easy to apply to the lips, does not run and does not migrate.

When it is applied to the cheeks, a comfortable, non-tacky coloured deposit is obtained.

The gloss of the deposit obtained immediately after application of the composition is satisfactory.

The deposit is fresh, very thin and comfortable, with no sensation of dryness and no tackiness.

It has good colour persistence on the lips and good transfer resistance.

### Protocol for Measuring the Tack:

The composition is deposited in several stainless steel dishes 100 μm deep and is levelled off as quickly as possible. The dishes are left to dry at room temperature for one hour.

The apparatus used is a TAXT2i texturometer. The clip mounted on the apparatus grips an AU4G cylinder 6 mm in diameter at the end of which is attached a smooth beige-coloured end piece made of synthetic skin, which has the same diameter and is 2 mm thick.

The end piece is cleaned with ethanol between each measurement.

Several measurements are never taken at the same place of the deposit.

The parameters of the compression tests with maintenance over time are indicated below:

| Approach speed (or pre-speed) | 1 | mm/s |
|---|---|---|
| Speed (starting from detection of the contact) | 0.1 | mm/s |
| Force (and corresponding pressure) | 0.283 N (i.e. 0.01 MPa) | |
| Maintenance time | 3 | s |
| Withdrawal speed (or post-speed) | 0.1 | mm/s |

The tackiness is characterized by the separation force measured during the pressure reduction (pull phase), corresponding to the integral of the curve under the time axis. This force is expressed positively in joules per square metre.

### Examples 2

The compositions whose ingredients are listed in the table below is prepared (the percentages are expressed as weight of starting material, unless otherwise indicated).

| | | Compar- |
|---|---|---|
| Ingredients (INCI name) | invention | ative |
| Polysorbate 60 (Tween 60-LQ-(MV) from Croda) | 4% | 4% |
| Sodium dehydroacetate (Tristat SDHA FCC/NF | 0.5% | 0.5% |
| from TRI-K) | | |
| Red 33 | 0.30% | 0.30% |
| Ammonium polyacryloyldimethyl taurate | 0.50% | 0.50% |
| (Hostacerin AMPS from Clariant) | | |
| Silicone resin (trimethyl siloxysilicate (and) | 44.5% | 11.67% |
| isododecane: X-21-5616 from Shin-Etsu; in a | | |
| silicone resin/isododecane weight ratio of 60/40) | | |
| Diphenylsiloxyphenyl trimethicone (KF56A from | 20% | 20% |
| Shin-Etsu) | | |
| Water | qs 100% | qs 100% |

### Procedure

The polysorbate 60 and water are heated at 50° C. with a deflocculator until the mixture is homogeneous. The heating is stopped.

The dye is then added and the mixture is stirred until dissolution is complete.

The thickening polymer is then added with stirring until the mixture thickens without gel lumps.

The silicone resin and the isododecane are then poured slowly onto the mixture obtained previously while increasing the stirring speed, and stirring is continued for 10 minutes.

Finally, the diphenylsiloxyphenyl trimethicone is added slowly and stirring is maintained for 10 minutes.

### Evaluation of the Compositions

Composition According to the Invention:

A stable, homogeneous direct emulsion is obtained. No phase separation is observed after centrifugation of the composition for 1 hour at 900×g according to the protocol detailed in the description.

The composition is easy to apply, does not run, does not migrate and does not stain the teeth.

When it is applied to the cheeks, a comfortable, non-tacky coloured deposit is obtained.

The gloss of the deposit obtained immediately after application of the composition is satisfactory.

The deposit is fresh, very thin and comfortable, with no sensation of dryness and no tackiness.

It has good colour persistence on the lips and good transfer resistance.

Comparative Composition

An unstable direct emulsion is obtained (separation of phases one day after preparation; three phases observed after centrifugation of the composition for 1 hour at 900×g according to the protocol detailed in the description).

The composition is difficult to apply homogeneously.

The sensation of comfort of the comparative composition disappears rapidly in comparison with the compositions according to the invention. The wear of color is also limited

### Example 3

The composition whose ingredients are listed in the table below is prepared (the percentages are expressed as weight of starting material, unless otherwise indicated).

| Ingredients (INCI name) | Content |
|---|---|
| MQ silicone resin in emulsion form (KM-9717 from Shin- | 45% |
| Etsu) | |
| Red 33 | 0.30% |
| Ammonium polyacryloyldimethyl taurate (Hostacerin AMPS | 0.50% |
| from Clariant) | |
| PEG-8 isostearate (Cithrol 4MIS-LQ-(GD) from Croda) | 2.7% |
| PEG-10 dimethicone (KF-6017 from Shin-Etsu) | 2.3% |
| Trimethylsiloxyphenyl Dimethicone (Belsil PDM 1000 from | 18% |
| Wacker) | |
| Water | qs 100% |

### Procedure

The emulsion KM-9717 and water are mixed with a deflocculator for 10 minutes.

The dye is then added and the mixture is stirred until dissolution is complete.

The thickening polymer is then added with stirring until the mixture thickens without gel lumps.

In a separate container, the fatty phase is prepared by heating the trimethylsiloxyphenyl dimethicone and the surfactants at 50° C. with stirring using a deflocculator until homogenization is complete, and the heating is then stopped.

Once the mixture is at room temperature, the fatty phase is poured slowly onto the aqueous phase while increasing the stirring speed, and stirring is continued for 10 minutes.

### Evaluation of the Composition

A stable, homogeneous direct emulsion is obtained.

No phase separation is observed after centrifugation of the composition for 1 hour at 900×g according to the protocol detailed in the description, a 1 mm supernatant at the surface.

The composition is easy to apply, does not run, does not migrate or migrates very little, and does not stain the teeth.

When it is applied to the cheeks, a comfortable, non-tacky coloured deposit is obtained.

The gloss of the deposit obtained immediately after application of the composition is satisfactory.

The deposit is fresh, very thin and comfortable, with no sensation of dryness and no tackiness.

It has good colour persistence on the lips and good transfer resistance.

1. A cosmetic composition comprising:
at least 10% by weight, relative to the weight of the composition, of water;
at least 10% by weight, relative to the weight of the composition, of at least one silicone resin;
at least one non-volatile silicone oil, not comprising any nitrogen atoms, or any —Si—H groups, or any (poly)oxyalkylene groups, the oxyalkylene unit being C₂-C₃, or any (poly)glycerol groups;
optionally at least one first non-volatile hydrocarbon-based oil in a content of less than 15% by weight, relative to the weight of the composition; said non-volatile hydrocarbon-based oil(s) being selected from the group consisting of:
C₁₀-C₂₆ alcohols;
optionally hydroxylated monoesters, diesters or triesters of a C₂-C₈ monocarboxylic or polycarboxylic acid and of a C₂-C₈ alcohol, which are optionally hydroxylated;
esters of a C₂-C₈ polyol and of one or more C₂-C₈ carboxylic acids;
optionally, at least one volatile oil in a content such that the silicone resin/volatile oil(s) weight ratio is greater than or equal to 1.

2. The composition according to claim 1, wherein the composition comprises a water content of between 10% and 70% by weight relative to the weight of the composition.

3. The composition according to claim 1, wherein the silicone resin is chosen selected from the group consisting of: —silicone resins of MQ type, such as the alkyl siloxysilicates especially of formula [(R1)₃SiO_{1/2}]_{X}(SiO_{4/2})_{y} (units MQ) in which x and y are integers ranging from 50 to 80, and such that the group R1 represents a hydrocarbon-based radical, especially alkyl, containing from 1 to 10 carbon atoms, a phenyl group, a phenylalkyl group or else a hydroxyl group,
silicone resins of T type, such as the polysilsesquioxanes in particular of formula (RSiO_{3/2})ₓ (units T) in which x is greater than 100 and such that the group R is an alkyl group containing from 1 to 10 carbon atoms, said polysilsesquioxanes also possibly comprising Si—OH end groups,
resins of MQT type, in particular MQT-propyl resins, comprising the following units:
(i) (R1₃SiO_{1/2})ₐ
(ii) (R2₂SiO_{2/2})_{b}
(iii) (R3SiO_{3/2})_{c} and
(iv) (SiO_{4/2})_{d}
with
R1, R2 and R3 independently representing a hydrocarbon-based radical, containing from 1 to 10 carbon atoms, a phenyl group, a phenylalkyl group or a hydroxyl group,
a, b, c and d being mole fractions,
a being between 0.05 and 0.5,
b being between zero and 0.3,
c being greater than zero,
d being between 0.05 and 0.6,
a+b+c+d=1,
with the proviso that more than 40 mol % of the groups R3 of the siloxane resin are propyl groups.

4. The composition according to claim 3, wherein the silicone resin is a resin of MQ type, and the resin is a siloxysilicate resin.

5. The composition according to claim 1, wherein said silicone resin is present in a content of between 10% and 45% by weight relative to the weight of the composition.

6. The composition according to claim 1, wherein the silicon resin is used in a powder form; in a form conveyed in a solvent, chosen from volatile or non-volatile apolar hydrocarbon-based oils and silicone oils; or in a form emulsified in water.

7. The composition according to claim 1, wherein the comprises a non-volatile silicone oil(s) which are selected from non-phenyl non-volatile silicone oils and phenyl non-volatile silicone oils, optionally bearing a dimethicone fragment.

8. The composition according to claim 7, wherein the non-volatile silicone oil(s) are chosen from polydimethylsiloxanes; phenyl non-volatile silicone oils bearing at least one dimethicone fragment, and also mixtures thereof.

9. The composition according to the preceding claim 8, wherein phenyl non-volatile silicone oil(s) are selected from the group consisting of: trimethylsiloxyphenyl dimethicones and diphenyl dimethicones, and mixtures thereof.

10. The composition according to claim 8, wherein the content of non-volatile silicone oil(s) represents from 2% to 35% by weight relative to the weight of the composition.

11. The composition according to claim 1, wherein the content of first non-volatile hydrocarbon-based oil(s) ranges from 0.5% to less than 15% by weight relative to the weight of the composition.

12. The composition according to claim 1, wherein the content of volatile hydrocarbon-based oil(s) is less than 30% by weight relative to the weight of the composition relative to the weight of the composition.

13. The composition according to claim 1, wherein the composition comprises at least one second polar or apolar non-volatile hydrocarbon-based oil, in a content such that the non-volatile silicone oil(s)/second non-volatile hydrocarbon-based oil(s) weight ratio is greater than 1.

14. The composition according to claim 1, wherein the composition further comprises at least one nonionic hydrocarbon-based surfactant with an HLB of greater than or equal to 8.

15. The composition according to claim 14, wherein the nonionic hydrocarbon-based surfactant(s) are selected from the group consisting of: alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, alkyl and polyalkyl esters of sucrose, optionally (poly)oxyethylenated alkyl and polyalkyl esters of glycerol, and optionally (poly)oxyethylenated alkyl and polyalkyl ethers of glycerol, and mixtures thereof.

16. The composition according to claim 14, wherein it comprises, as additional surfactant(s), at least one nonionic hydrocarbon-based surfactant whose HLB value is less than 8; at least one nonionic silicone surfactant, with an HLB value of less than 8; and also mixtures thereof.

17. Composition The composition according to claim 14, wherein wherein the content of nonionic surfactant(s) represents from 0.1% to 15% by weight relative to the weight of the composition.

18. The composition according to claim 1, wherein the composition comprises at least one anionic hydrocarbon-based surfactant selected from the group consisting of: salts of alkali metals, of amine or of alkanolamine, of fatty acids especially of C₈-C₂₀, in a content of not more than 2.5% by weigh relative to the weight of the composition.

19. The composition according to claim 1, wherein the composition comprises at least one hydrophilic thickening polymer, selected from the group consisting of: homopolymers or copolymers of acrylic or methacrylic acid or salts thereof and esters thereof; copolymers of acrylic acid and of acrylamide; homopolymers and copolymers based on acrylamidopropanesulfonic acid; anionic, cationic, amphoteric or nonionic chitin or chitosan polymers; cellulose polymers; vinyl polymers; optionally modified polymers of natural origin; alginates and carrageenans; mucopolysaccharides, and mixtures thereof.

20. The composition according to claim 19, wherein the content of hydrophilic thickener is between 0.01% and 1.5% by weight relative to the weight of the composition.

21. The composition according to claim 1, wherein the composition comprises at least one dyestuff, selected from the group consisting of: pigments, nacres, water-soluble dyes and liposoluble dyes, and also mixtures thereof.

22. The composition according to claim 1, wherein the composition is in the form of an oil-in-water emulsion.

23. A process for making up and/or caring for the skin, comprising: applying the composition according to claim 1 to the skin.
